(19) United States
(12) Patent Application Publication
(10) Pub. No.: US 2024/0115556 A1
(43) Pub. Date: Apr. 11, 2024

(54) A JMJD6 TARGETING AGENT FOR TREATING PROSTATE CANCER

(71) Applicants: Cancer Research Technology Limited, London (GB); The Chancellor, Masters And Scholars Of The University Of Oxford, Oxford (GB); The Institute of Cancer Research: Royal Cancer Hospital, London (GB); University of Washington, Seattle, WA (US); The United States Government represented by The Department Of Veterans Affairs, Washington, DC (US)

(72) Inventors: Md. Saiful Islam, Oxford (GB); Anthony Tumber, Oxford (GB); Christopher Schofield, Oxford (GB); Alec Paschalis, London (GB); Jonathan Welti, London (GB); Adam Sharp, London (GB); Johann de Bono, London (GB); Stephen Plymate, Seattle, WA (US)

(21) Appl. No.: 18/257,801

(22) PCT Filed: Dec. 17, 2021
(86) PCT No.: PCT/GB2021/053349

§ 371 Date: Jun. 15, 2023

Related U.S. Application Data
(60) Provisional application No. 63/126,812, filed on Dec. 17, 2020.

(51) Int. Cl. A61K 31/44; C12N 15/113; C12Q 1/6886

(52) U.S. Cl. CPC A61K 31/44; C12N 15/1137; C12Q 1/6886; C12N 2310/14

(57) ABSTRACT

The invention relates to methods for treating prostate cancer by targeting the generation of splice variants of the androgen receptor. In one aspect, this can be achieved by targeting JMJD6 to reduce the production of androgen receptor splice variants. The invention finds particular use in the treatment of prostate cancer that is resistant to conventional androgen therapy.

### STATEMENT REGARDING FEDERALLY SPONSORED RESEARCH

This invention was made with US government support under Grant No. W81XWH-17-1-0323, awarded by the Department of Defense. The government has certain rights in the invention.

### FIELD OF THE INVENTION

The invention relates to methods for treating prostate cancer by targeting the generation of splice variants of the androgen receptor. The invention also relates to associated compositions, uses and methods.

### INTRODUCTION

Prostate cancer (PC) is a leading cause of male cancer mortality globally. PC progression to metastatic castration-resistant PC (mCRPC) is commonly driven by persistent androgen receptor (AR) signaling [1, 2]. Abiraterone and enzalutamide, which target the AR signaling axis, are standards of care, improving both progression free (PFS) and overall survival (OS) [3, 4] for mCRPC and castration-sensitive PC (CSPC) [5]. However, some mCRPCs have minimum response to these therapies, while all eventually acquire resistance and invariably cause fatality [6], in part, due to constitutively active alternatively spliced AR variants (AR-SVs) that are truncated and lack the regulatory AR ligand-binding domain targeted by current AR directed therapies [7-9]. Of the AR-SVs reported, AR splice variant 7 (AR-V7) is particularly prevalent and is associated with resistance to AR targeting therapies and poorer OS [8, 10]. Efforts to target AR-SVs directly have proved challenging due to the inherently disordered nature of the AR N-terminal domain [9].

One strategy to abrogate AR-V7-mediated resistance is to target processes regulating AR-V7 generation and/or stabilization. Members of the bromodomain and extra-terminal (BET) motif protein family are of interest as they are reported to modulate AR signaling [11]; BET inhibition downregulates AR-V7 protein expression and reduces enzalutamide-resistant patient-derived PC model growth [11]. However, BET proteins have pleiotropic roles and regulate many signaling pathways, perhaps explaining why despite extensive efforts no BET inhibitors have not yet been approved for clinical use [12].

There remains an unmet need for novel therapeutic strategies to overcome AR-SVs and improve outcome from lethal PC. The invention is aimed at addressing this need.

### SUMMARY OF THE INVENTION

Endocrine resistance (EnR) in advanced prostate cancer (APC) is fatal. EnR can be mediated by androgen receptor splice variants (AR-SV), with AR-V7 a particularly important clinical variant. The present inventors have determined proteins which are key to generating AR-V7. JMJD6 was identified as a key regulator of AR-V7, as evidenced by its upregulation with in vitro EnR, its downregulation alongside AR-V7 by bromodomain inhibition, and its identification in targeted siRNA screen of spliceosome-related genes. JMJD6 protein levels increased (p<0.001) with castration resistance and were associated with higher AR-V7 levels and shorter survival (p=0.048). JMJD6 knockdown reduced PC cell growth, AR-V7 levels, and recruitment of U2AF65 to AR pre-mRNA. The present inventors have also shown that knock-down of the JMJD6 gene and inhibition of the JMJD6 protein result in reduction of AR-V7 levels and as such targeting of JMJD6 may be key to reducing prostate cancer cell growth.

As such in a first embodiment the invention relates to a JMJD6 targeting agent for use in the treatment or prevention of prostate cancer.

In an aspect the invention relates to a pharmaceutical composition comprising a JMJD6 targeting agent for use in the treatment of prostate cancer.

In an aspect the present invention relates to a kit comprising a JMJD6 targeting agent or a pharmaceutical composition comprising a JMJD6 targeting agent and instructions for use.

In an aspect the present invention relates to a method of diagnosing or prognosing prostate cancer, comprising
a. obtaining a biological sample,
b. determining the level of JMJD6 in the sample
   wherein an increased level of JMJD6 compared to a reference sample indicates a poor prognosis.

In another aspect, the invention relates to a method of inhibiting androgen receptor splicing comprising contacting a cell with a JMJD6 targeting agent. Thus, the invention also relates to reducing generation of androgen receptor splice variants such as AR-V7.

In another aspect, the invention relates to a pharmaceutical composition comprising an androgen therapy and a JMJD6 targeting agent as defined herein. This can be used in the treatment of prostate cancer, in particular prostate cancer that is resistant to conventional androgen therapy.

In an aspect the invention relates to a method of monitoring the therapeutic efficacy of a prostate cancer therapy, comprising determining the level of JMJD6 prior to administration of the therapy and determining the level of JMJD6 after administration of the therapy.

In an aspect the invention relates to a method of identifying a JMJD6 targeting agent, comprising contacting a cell with a compound and determining the level of androgen receptor splicing.

### FIGURES

The invention is further illustrated in the following non-limiting figures.

FIG. 1: Orthogonal analyses identify the 2OG-dependent dioxygenase JMJD6 as a potential regulator of AR-V7. (A) Volcano plots illustrating differential mRNA expression of 315 genes relating to the spliceosome (spliceosome related gene set;), as determined by RNA-seq, between hormone-sensitive LNCaP (no AR-V7 protein) and androgen deprivation resistant LNCaP95 (detectable AR-V7 protein) prostate cancer (PC) cell lines, and LNCaP95 PC cells treated with either a BET inhibitor (I-BET151) or vehicle (DMSO 0.1%). Blue dots represent genes with baseline expression (FPKM) greater than the median expression level of all 315 genes at baseline across both experiments. Top 15 genes most differentially expressed (FPKM) in each experiment (up- or down-regulated) indicated by red dots. Top 10 hits identified in targeted siRNA screen shown in accompanying table; all 315 genes in the spliceosome related gene set were individually inhibited by siRNA in 22Rv1 and LNCaP95 PC cell lines. Changes in AR-V7 protein levels relative to AR-FL were quantified by western blot (WB) densitometry. AR-V7 downregulation averaged across both cell lines with genes ranked in order of the degree of AR-V7 downregulation relative to AR-FL. (B) Venn diagram amalgamating RNA-seq analyses with siRNA screen results. Genes of interest pre-defined as being upregulated in LNCaP95 cells relative to LNCaP cells, downregulated following BET inhibition, and associated with a >50% reduction in AR-V7 protein expression (WB) relative to AR-FL following siRNA knockdown. JMJD6 was the only gene to meet all three criteria. (C-E) Scatter plots of transcriptome analysis in 159 mCRPC biopsies (SU2C/PCF cohort) showing correlations between JMJD6 mRNA expression and (C) androgen response (Hallmark; H), (D) AR signature (derived from 43 AR regulated transcripts) and (E) AR-V7 signature (derived from 59 genes associated with AR-V7 expression in mCRPC). JMJD6 mRNA expression shown as log FPKM. r-values and p-values are shown and were calculated using Spearman's correlation.

FIG. 2: JMJD6 associates with AR-V7 expression and a worse prognosis in mCRPC. (A) Antibody specificity confirmed by detection of a single band in LNCaP95 whole cell lysates by WB, with downregulation following treatment with pooled JMJD6 siRNA compared to non-targeting control siRNA. (B) Micrograph of LNCaP95 PC cells treated with non-targeting control siRNA demonstrating positive brown nuclear staining for JMJD6. (C) Micrograph of LNCaP95 PC cells treated with pooled JMJD6 siRNA. Demonstrates a marked reduction in JMJD6 protein, with predominately blue, negative staining for JMJD6. (D) Micrographs of IHC analyses for AR-V7 (left) and JMJD6 (right) protein levels in matched, same-patient, diagnostic castration-sensitive (CSPC) (top) and mCRPC (bottom) tissue samples from three different patients (RMH/ICR patient cohort). Scale bars set to 100 m. JMJD6 protein levels in presented tissue samples are similar to AR-V7 levels in mCRPC. (E) Box and whisker plot demonstrating a significant increase (p<0.001) in JMJD6 protein levels (IHC H-Score) in mCRPC biopsies (median H-score [IQR]; CSPC (n=64) 12.5 [0.0-67.5] vs CRPC (n=74) 80 [20.0-130.0]; Wilcoxon rank-sum analysis). (F) Levels of AR-V7 protein significantly higher (p=0.036) in mCRPC tissue samples from patients with high (JMJD6 H-Score≥median) mCRPC JMJD6 protein levels (Low 50 [0.0-105.0; n=33] vs High 100 [22.5-147.5; n=41]; Mann-Whitney test). (G) Median OS from the time of CRPC tissue biopsy significantly worse in patients with the highest levels of JMJD6 (H-Score>75^{th} percentile) in their mCRPC tissue sample (n=74, p=0.048; Log-rank test).

FIG. 3: JMJD6 is important for PC cell growth and regulates AR-V7 expression. (A) JMJD6 siRNA knockdown (25 nM; red bars/right-hand bars) significantly reduces the growth (cell number; sulforhodamine B (SRB) assay) of LNCaP, LNCaP95 and 22Rv1 PC cells compared to non-targeting control siRNA (25 nM; blue bars/left-hand bars), while PNT2 cells (immortalized normal prostatic epithelial cells) were relatively unaffected. Mean cell growth (normalized to control siRNA at same concentration) shown with standard error of the mean; n≥4 data points (at least 2 biological replicates with 2 technical replicates). (B-C) JMJD6 siRNA knockdown downregulates AR-V7 mRNA (qPCR) and protein (WB) levels in LNCaP95 and 22Rv1 PC cell lines. Mean RNA expression (normalized to housekeeping genes (B2M and GAPDH) and control siRNA at equivalent concentration; defined as 1.0) with standard error of the mean from three experiments is shown. Control siRNA is shown in the left-hand bars and JMJD6 siRNA is shown in the right-hand bars (D) Line graph illustrating the impact of JMJD6 siRNA knockdown (25 nM)+/−enzalutamide (10M) on the viability of hormone-sensitive, AR amplified and AR-V7 producing VCaP PC cells compared to controls after five days, as determined using the CellTiter-Glo® Luminescent Cell Viability Assay. JMJD6 siRNA knockdown (red line) significantly reduced VCaP PC cell viability compared to control siRNA (blue line). Combination treatment with enzalutamide (purple line) resulted in a significantly more profound reduction of VCaP cell viability than either JMJD6 siRNA alone (red) or enzalutamide alone (green). n=3; mean cell viability (normalized to control siRNA at same concentration+DMSO 0.1%) shown with standard error of the mean. (E) JMJD6 knockdown downregulated baseline AR-V7 mRNA (qPCR) levels in VCaP cells. JMJD6 knockdown also resulted in a significantly lower increase in AR-V7 mRNA expression in response to AR blockade (enzalutamide 10M; purple bar/fourth bar from left to right) compared to non-targeting control siRNA (green bar/second bar from left to right). Mean RNA expression (normalized to housekeeping genes (B2M, GAPDH and CDC73), and control siRNA at equivalent concentration+DMSO 0.1%; defined as 1.0, blue bar first form the left) with standard error of the mean from three experiments is shown. (F) Single representative WB shown from three separate experiments. JMJD6 siRNA knockdown reduces AR-V7 protein levels in VCaP PC cells. Furthermore, while AR-V7 protein levels increase significantly with AR blockade (enzalutamide 10M), AR-V7 protein levels do not significantly change when JMJD6 is knocked down by siRNA (25 nM) at the time of treatment with enzalutamide (10M). p values (*, p≤0.05; **, p≤0.01; ***, p≤0.001) were calculated for each condition compared to control (at equivalent concentration) using the mean value of technical replicates with unpaired Student's t tests.

FIG. 4: JMJD6 regulates AR-V7 transcription, in part, through recruitment of splicing factor U2AF65 to AR-V7 specific splice sites in in vitro models of CRPC. (A-C) Scatter plots showing correlations between JMJD6 mRNA expression and (A) androgen response (Hallmark; H), (B) AR signature (derived from 43 AR regulated transcripts) and (C) AR-V7 signature (derived from 59 genes associated with AR-V7 expression in mCRPC) in 159 mCRPC biopsies (SU2C/PCF cohort). U2AF65 mRNA expression shown as log FPKM. r-values and p-values are shown and were calculated using Spearman's correlation. (D) Single WB in technical triplicate demonstrating reduction in AR-V7 protein levels with both JMJD6 and U2AF65 siRNA in 22Rv1 PC cells. JMJD6 siRNA had minimal impact on U2AF65 protein levels. (E) Schematic diagram of the human AR gene illustrating the regions targeted in RNA immunoprecipitation (RIP) assay with accompanying summary bar chart. Shows a reduction in detectable U2AF65 at the AR-V7 specific splice sites P1 (containing the 5′ splice site for both AR and AR-V7) and P2 (containing the 3′ splice site for AR-V7) in 22Rv1 PC cells treated with JMJD6 siRNA (shown in red bars/right-hand bars) compared to non-targeting control siRNA (shown in blue bars/left-hand bars. Indicates that JMJD6 regulates recruitment of the splicing factor U2AF65 to AR-V7 splice sites. RIP data derived from two independent experiments conducted in triplicate. p values (*, p≤0.05; **, p≤0.01; ***, p≤0.001) were calculated for each condition compared to control (at equivalent concentration) using the mean value of technical replicates with unpaired Student's t tests. (F) Schematic representation of alternative splicing events alongside corresponding histogram of alternative splicing mean differences between non-targeting control siRNA (blue dotted line; defined as 0.0) and JMJD6 siRNA in LNCaP95 PC cells. Left shift denotes decrease in splicing events. Total number of alternative splicing events (x) occurring in total number of genes (y) shown in orange (x/y). JMJD6 knockdown led to substantial changes in 753 alternative splicing events, with the majority of these occurring less frequently.

FIG. 5: Evidence JMJD6-mediated AR-V7 generation is dependent on JMJD6 catalysis, which can be chemically inhibited to downregulate AR-V7 protein levels. (A) Transfection of a JMJD6 wild-type (JMJD6^{WT}) plasmid at increasing concentrations (all receiving 1 g of plasmid in total, with empty vector control added to make up the difference) into 22Rv1 PC cells led to an increase in AR-V7 protein (WB) and mRNA (qPCR) levels. Mean mRNA levels were normalized to housekeeping genes (B2M and GAPDH), and to studies with an empty vector control plasmid at equivalent concentration; the empty vector control data were defined as 1.0 with standard error of the mean from three experiments shown. p values (*, p≤0.05;**, p≤0.01; ***, p≤0.001) were calculated for each condition compared to control (at equivalent concentration), using the mean value of technical replicates with unpaired Student's t tests. (B) Conversely, transfection with inactivating mutations of active site residues in the JMJD6 catalytic domain by JMJD6^{MUT1} (D189A and H187A) and JMJD6^{MUT2} (N287A and T285A) decreased AR-V7 protein levels (empty vector control, JMJD6^{MUT1} and JMJD6^{MUT2}=1 g of total plasmid). (C) AR-V7 expression was induced by JMJD6^{WT} but not by JMJD6^{MUT1} in VCaP PC cells, suggesting that JMJD6-mediated AR-V7 expression requires active JMJD6. Singleton WB validating findings presented in (B) in an alternative cell line model. (D-E) Graphic representation of JMJD6 tertiary structure [58]. The inactivating substitutions of active site residues in the JMJD6 catalytic domain by JMJD6^{MUT1} (D189A and H187A; green spheres) and JMJD6^{MUT2} (N287A and T285A; magenta spheres) reside within a predicted druggable pocket (shown in orange), identified by the canSAR knowledgebase [33, 34]. (F) Liquid chromatography-mass spectrometry (LC-MS) analysis demonstrating that the 2OG mimic pyridine-2,4-dicarboxylic acid (2,4-PDCA) resulted in a dose-dependent reduction in isolated JMJD6-mediated lysyl-5-hydroxylation of its known target LUC7L; indicating that 2,4-PDCA is an inhibitor of JMJD6 lysyl hydroxylase catalytic activity. (G) WB showing that 2,4-PDCA caused a dose-dependent reduction of AR-V7 protein levels in 22Rv1 PC cells. Single representative WB shown from two separate experiments

### DETAILED DESCRIPTION OF THE INVENTION

The embodiments of the invention will now be further described. In the following passages, different embodiments are described. Each aspect so defined may be combined with any other aspect or aspects unless clearly indicated to the contrary.

In particular, any feature indicated as being preferred or advantageous may be combined with any other feature or features indicated as being preferred or advantageous. The practice of the present invention will employ, unless otherwise indicated, conventional techniques of immunology, molecular biology, medicinal chemistry, enzymology including relating to 2-oxoglutarate dependent oxygenases and their inhibition, biochemistry and recombinant DNA technology, which are within the skill of the art. Molecular biology techniques are explained fully in the literature, see, e.g., Green and Sambrook et al., Molecular Cloning: A Laboratory Manual, 4th ed., Cold Spring Harbor Laboratory Press, Cold Spring Harbor, N.Y. (2012).

Androgen receptor signaling is critical for prostate development and progression. Constitutively active AR splice variants, such as AR-V7, induce resistance to anti-androgen therapies, such as abiraterone acetate (AA), enzalutamide (E), and apalutamide, that target the AR axis in patients with castration-sensitive prostate cancer (CSPC) and castration-resistant prostate cancer CRPC. As such there is a need for therapies which can reduce the production of AR splice variants.

The present invention is based on the finding that JMJD6 is a key protein in androgen receptor splicing, e.g. in the generation of AR-V7. As such the present inventors have found that the production of androgen receptor splice variants, such as AR-V7, can be reduced or prevented by targeting JMJD6.

Thus, the invention relates to a JMJD6 targeting agent for use in the treatment of prostate cancer. This is achieved by preventing the production of one or more androgen receptor splice variant, e.g. AR-V7, through targeting JMJD6.

The terms “JMJD6” or “JMJD6 sequence” as used herein may refer to the gene or a nucleic acid encoding Jumonji Domain-Containing Protein 6 or may refer to the Jumonji Domain-Containing Protein 6 itself (Uniprot accession Q6NYC1), including any variants/isoforms of JMJD6, e.g. as occur by post-translational modification. The NCBI ID for isoform one of JMJD6 is NP_001074930.1, and isoform two of JMJD6 is NP_055982.2. Thus, as used herein in the various embodiments of the invention, the JMJD6 nucleic acid sequence may comprise SEQ ID NO. 1 (isoform 1) or SEQ ID NO. 3 (isoform 2) or part thereof. The JMJD6 polypeptide sequence may comprise SEQ ID NO. 3 (isoform 1) or SEQ ID NO. 4 (isoform 2) or part thereof. In some embodiments, the JMJD6 nucleic acid or polypeptide sequence is a variant or truncated form (e.g. N or C terminally truncated, e.g. by 5, 10, 15 or 20 residues) of a nucleic acid or polypeptide sequence provided herein, e.g. having a sequence with a sequence homology or identity of at least 85%, preferably of at least 90%, more preferably of at least 95%, still more preferably of at least 98% with the relevant nucleic acid or polypeptide sequence of JMJD6, e.g. as defined in SEQ ID NO. 1, 2, 3 or 4.

SEQ ID NO. 1
ATGAACCACAAGAGCAAGAAGCGCATCCGCGAGGCCAAGCGGAGTGCGCGGCCGGAGC
TCAAGGACTCGCTGGATTGGACCCGGCACAACTACTACGAGAGCTTCTCGCTGAGCCCG
GCGGCCGTGGCGGATAACGTGGAAAGGGCAGATGCTTTACAGCTGTCTGTGGAAGAATT
TGTGGAGCGGTATGAAAGACCTTACAAGCCCGTGGTTTTGTTGAATGCGCAAGAGGGCT
GGTCTGCGCAGGAGAAATGGACTCTGGAGCGCCTAAAAAGGAAATATCGGAACCAGAAG
TTCAAGTGTGGTGAGGATAACGATGGCTACTCAGTGAAGATGAAGATGAAATACTACATC
GAGTACATGGAGAGCACTCGAGATGATAGTCCCCTTTACATCTTTGACAGCAGCTATGGT
GAACACCCTAAAAGAAGGAAACTTTTGGAAGACTACAAGGTGCCAAAGTTTTTCACTGATG
ACCTTTTCCAGTATGCTGGGGAGAAGCGCAGGCCCCCTTACAGGTGGTTTGTGATGGGG
CCACCACGCTCCGGAACTGGGATTCACATCGACCCTCTGGGAACCAGTGCCTGGAATGC
CTTAGTTCAGGGCCACAAGCGCTGGTGCCTGTTTCCTACCAGCACTCCCAGGGAACTCAT
CAAAGTGACCCGAGACGAAGGAGGGAACCAGCAAGACGAAGCTATTACCTGGTTTAATG
TTATTTATCCCCGGACACAGCTTCCAACCTGGCCACCTGAATTCAAACCCCTGGAAATCTT
ACAAAAACCAGGAGAGACTGTCTTTGTACCAGGAGGCTGGTGGCATGTTGTCCTCAATCT
CGACACTACTATCGCCATCACCCAAAATTTTGCCAGCAGCACCAACTTCCCTGTGGTATG
GCACAAGACGGTAAGAGGGAGACCAAAGTTATCAAGGAAATGGTATAGGATTTTGAAGCA
AGAGCACCCCGAGTTGGCAGTCCTCGCAGACTCGGTTGACCTTCAGGAGTCCACAGGGA
TAGCTTCCGACAGCTCCAGCGACTCTTCCAGCTCCTCCAGCTCCAGTTCGTCAGACTCCG
ACTCAGAGTGCGAGTCTGGATCCGAGGGCGATGGGACAGTGCACCGCAGGAAGAAGAG
GAGGACGTGCAGCATGGTGGGAAACGGGGACACCACCTCCCAGGACGACTGTGTCAGC
AAAGAGCGCAGCTCCTCCAGGATTAGGGACACTTGTGGAGGCCGGGCTCACCCCTGA
SEQ ID NO. 2
MNHKSKKRIREAKRSARPELKDSLDWTRHNYYESFSLSPAAVADNVERADALQLSVEEFVER
YERPYKPVVLLNAQEGWSAQEKWTLERLKRKYRNQKFKCGEDNDGYSVKMKMKYYIEYME
STRDDSPLYIFDSSYGEHPKRRKLLEDYKVPKFFTDDLFQYAGEKRRPPYRWFVMGPPRSGT
GIHIDPLGTSAWNALVQGHKRWCLFPTSTPRELIKVTRDEGGNQQDEAITWFNVIYPRTQLPT
WPPEFKPLEILQKPGETVFVPGGWWHVVLNLDTTIAITQNFASSTNFPVVWHKTVRGRPKLS
RKWYRILKQEHPELAVLADSVDLQESTGIASDSSSDSSSSSSSSSSDSDSECESGSEGDGTV
HRRKKRRTCSMVGNGDTTSQDDCVSKERSSSRIRDTCGGRAHP
SEQ ID NO. 3
ATGAACCACAAGAGCAAGAAGCGCATCCGCGAGGCCAAGCGGAGTGCGCGGCCGGAGC
TCAAGGACTCGCTGGATTGGACCCGGCACAACTACTACGAGAGCTTCTCGCTGAGCCCG
GCGGCCGTGGCGGATAACGTGGAAAGGGCAGATGCTTTACAGCTGTCTGTGGAAGAATT
TGTGGAGCGGTATGAAAGACCTTACAAGCCCGTGGTTTTGTTGAATGCGCAAGAGGGCT
GGTCTGCGCAGGAGAAATGGACTCTGGAGCGCCTAAAAAGGAAATATCGGAACCAGAAG
TTCAAGTGTGGTGAGGATAACGATGGCTACTCAGTGAAGATGAAGATGAAATACTACATC
GAGTACATGGAGAGCACTCGAGATGATAGTCCCCTTTACATCTTTGACAGCAGCTATGGT
GAACACCCTAAAAGAAGGAAACTTTTGGAAGACTACAAGGTGCCAAAGTTTTTCACTGATG
ACCTTTTCCAGTATGCTGGGGAGAAGCGCAGGCCCCCTTACAGGTGGTTTGTGATGGGG
CCACCACGCTCCGGAACTGGGATTCACATCGACCCTCTGGGAACCAGTGCCTGGAATGC
CTTAGTTCAGGGCCACAAGCGCTGGTGCCTGTTTCCTACCAGCACTCCCAGGGAACTCAT
CAAAGTGACCCGAGACGAAGGAGGGAACCAGCAAGACGAAGCTATTACCTGGTTTAATG
TTATTTATCCCCGGACACAGCTTCCAACCTGGCCACCTGAATTCAAACCCCTGGAAATCTT
ACAAAAACCAGGAGAGACTGTCTTTGTACCAGGAGGCTGGTGGCATGTTGTCCTCAATCT
CGACACTACTATCGCCATCACCCAAAATTTTGCCAGCAGCACCAACTTCCCTGTGGTATG
GCACAAGACGGTAAGAGGGAGACCAAAGTTATCAAGGAAATGGTATAGGATTTTGAAGCA
AGAGCACCCCGAGTTGGCAGTCCTCGCAGACTCGGTTGACCTTCAGGAGTCCACAGGGA
TAGCTTCCGACAGCTCCAGCGACTCTTCCAGCTCCTCCAGCTCCAGTTCGTCAGACTCCG
ACTCAGAGTGCGAGTCTGGATCCGAGGGCGATGGGACAGTGCACCGCAGGAAGAAGAG
GAGGACGTGCAGCATGGTGGGAAACGGGGACACCACCTCCCAGGACGACTGTGTCAGC
AAAGAGCGCAGCTCCTCCAGGTGA
SEQ ID NO. 4
MNHKSKKRIREAKRSARPELKDSLDWTRHNYYESFSLSPAAVADNVERADALQLSVEEFVER
YERPYKPVVLLNAQEGWSAQEKWTLERLKRKYRNQKFKCGEDNDGYSVKMKMKYYIEYME
STRDDSPLYIFDSSYGEHPKRRKLLEDYKVPKFFTDDLFQYAGEKRRPPYRWFVMGPPRSGT
GIHIDPLGTSAWNALVQGHKRWCLFPTSTPRELIKVTRDEGGNQQDEAITWFNVIYPRTQLPT
WPPEFKPLEILQKPGETVFVPGGWWHVVLNLDTTIAITQNFASSTNFPVVWHKTVRGRPKLS
RKWYRILKQEHPELAVLADSVDLQESTGIASDSSSDSSSSSSSSSSDSDSECESGSEGDGTV
HRRKKRRTCSMVGNGDTTSQDDCVSKERSSSR

The JMJD6 protein is a nuclear localised protein (though it can be present elsewhere in cells) with a JmjC domain (Jumonji C domain). JMJD6 is a dioxygenase that has been reported as both an arginine demethylase and as a lysyl-hydroxylase. Catalytic activity of JMJD6 requires an Fe(II) as a cofactor and 2-oxoglutarate (2OG) and dioxygen as cosubstrates. Carbon dioxide and succinate are produced as coproducts.

As used herein the term “JMJD6 targeting agent” is any agent capable of targeting either the JMJD6 gene (including both DNA and RNA encoding for the JMJD6 protein) or the JMJD6 protein. Thus, in one embodiment, the “JMJD6 targeting agent” is any agent capable of targeting the JMJD6 gene. In another embodiment, the “JMJD6 targeting agent” is any agent capable of targeting the JMJD6 protein. The targeting agent may be an agent which inhibits or reduces the catalytic activity and/or biological function/activity of the JMJD6 protein or expression of the JMJD6 gene. The targeting agent may be an agent which modulates the catalytic activity and/or biological function/activity of the JMJD6 protein or the expression of the JMJD6 gene. Modulation of catalytic activity may comprise a reduction in the catalytic activity, variation in catalytic turn-over rate and/or alteration of substrate recognition.

In an embodiment the targeting agent may be a small molecule inhibitor or a biomacromolecule such as an antibody or a fragment thereof. Levels of JMJD6, e.g. levels JMJD6 gene expression, may also be altered by use of nucleic acids, e.g. short interfering RNA or CRISPR methodologies. The small molecule inhibitor or the antibody may bind in a manner which blocks the catalytic activity of JMJD6 protein. The small molecule inhibitor or the antibody may bind in a manner which reduces or substantially abolishes the catalytic activity of JMJD6 protein. Reduction of activity as used herein may be by 50%, 60%, 70%, 80%, 90% or more.

The JMJD6 targeting agent may be an inhibitor of the activity of JMJD6 protein, in particular with respect to its role in regulating levels of androgen splice variants such as AR V7. The JMJD6 targeting agent may bind in a manner which hinders the binding of the substrate or cosubstrate to the protein, for example the targeting agent may alter the conformation of the JMJD6 protein such that a substrate or cosubstrate can no longer bind, or it may block the binding or active site of the JMJD6 protein.

In one embodiment, the inhibitor reduces the catalytic activity of JMJD6 by binding to JMJD6. The JMJD6 targeting agent may act as a substrate or cosubstrate competitive inhibitor of the JMJD6 protein, as such the targeting agent may bind in a manner which targets the active site of JMJD6. The targeting agent may act as a competitive inhibitor with the substrate of JMJD6 and/or the cosubstrates 2OG or dioxygen. In particular, where the JMJD6 targeting agent is a competitive inhibitor, it may bind within the active site of JMJD6. The JMJD6 targeting agent may bind to the catalytic domain of JMJD6.

The competitive inhibitor may be selected from known inhibitors, or variants thereof, of human oxygenases such as the hypoxia inducible factor prolyl hydroxylases, clinically used inhibitors of which include active site Fe binding 2OG competitors, in particular Fe(II) binding 2OG competitors.

The term “active site” refers to the region of an enzyme such as JMJD6, wherein the region of the substrate undergoing a chemical reaction binds. Once the substrate is bound at the active site, catalysis occurs and the substrate is converted to the product. Catalytic residues within the active site act to lower the activation energy required for the conversion of substrate to product. The structure of the JMJD6 protein has been identified through X-ray crystallographic studies and the following residues have been identified as important catalytic residues which lie within a druggable pocket, these residues include; D189, H187, N287 and T285. As such, the JMJD6 targeting agent may block the interaction of the JMD6 substrate, cosubstrate, or metal ion, with active site residues, including but not limited to D189, H187, N287 and/or T285. In an embodiment the JMJD6 targeting agent may block the interaction of the Fe(II) cofactor with active site residues D189, H187, N287 and/or T285.

The JMJD6 targeting agent may be a non-substrate or non-co-substrate competitive JMJD6 inhibitor; as such the targeting agent may bind at a site away from the active site of JMJD6. The targeting agent may bind at a region of the JMJD6 protein, wherein such binding modulates activity of JMJD6, such as for example the polyserine region, the AT hook region, and/or the nuclear localisation region.

The JMJD6 targeting agent may be an uncompetitive inhibitor of JMJD6, as such the targeting agent may bind to the enzyme-substrate complex and prevent product formation. The JMJD6 targeting agent may be an allosteric inhibitor of JMJD6, as such the targeting agent may bind at a site away from the active site of JMJD6 and binding of the allosteric inhibitor may result in an altered protein conformation of JMJD6 such that the substrate cannot bind.

The inhibitor may compete with the substrate, cosubstrate or cofactor of JMJD6, processes which are all well established for the inhibition of human 2OG oxygenases. In particular the inhibitor may compete with the metal cofactor of JMJD6, Fe(II).

The JMJD6 targeting agent may comprise a compound which is an analogue or mimic of a 2OG (2-oxoglutarate, also known as α-ketoglutarate) or a compound that competes with 2OG. A mimic of 2OG may have structural or steric similarities to 2OG which has the following structure:

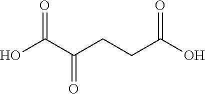

Examples of mimics of 2OG include pyridine-carboxylate derivatives or derivatives thereof, N-oxalyl amino acids or derivatives thereof, succinate or derivatives thereof, or 2OG or 2-oxo acid derivatives. In particular the mimic may be pyridine-2,4-dicarboxylic acid, as such the JMJD6 targeting agent may comprise or may consist of pyridine-2,4-dicarboxylic acid also known as lutidinic acid. It is recognised that 2OG competitors are well established as 2OG oxygenase inhibitors and are already used in medicine (e.g. hypoxia inducible factor prolyl hydroxylases inhibitors) and agriculture. Such inhibitors may or may not compete with the ‘prime’ enzyme substrate. Such already used compounds, or modifications or variants of them may be suitable for use as JMJD6 inhibitors for treatment of prostate cancer as described herein.

Known oxygenase inhibitors include FG4592 (Roxadustat), GSK1278863 (Daprodustat), Bay85-3934 (Molidustat), and AKB-6548 (Vadadustat). These compounds act by competing for binding with the cosubstrate 2OG (Yeh et al., Molecular and Cellular Mechanisms of HIF Prolyl Hydroxylase Inhibitors in Clinical Trials, Chem Sci, 2017, 8, 7651). As such, FG4592 (Roxadustat), GSK1278863 (Daprodustat), Bay85-3934 (Molidustat), and AKB-6548 (Vadadustat), or variants thereof, may be used to inhibit JMJD6 activity by competing for binding with 2OG and thereby reducing production of androgen receptor splice variants.

In mimics of 2OG, the acid groups at either end of the 2OG molecule may be replaced with a different functional group, for example a tetrazole, a triazole, alcohol group, a ketone, an aldehyde, an acyl-halide, a carboxylate, or an ester. Optimisation of 2OG mimics may be performed using standard drug discovery techniques and platforms to optimise binding and inhibitor characteristics.

The JMJD6 targeting agent may result in a reduction of the JMJD6 lysyl hydroxylase catalytic activity. It is also recognised that binding to JMJD6 may alter its biological function without altering its catalytic activity. For example, the JMJD6-mediated lysyl-5-hydroxylation of the target LUC7-Like (LUC7L) may be reduced.

The JMJD6 targeting agent may comprise a compound which competes for binding with the Fe(II) cofactor. As such the compound may bind in or close to the Fe(II) cofactor binding site such that the Fe(II) cannot bind.

The JMJD6 targeting compound may comprise a natural product or a known compound or a variant or prodrug form of these.

In one embodiment, the inhibitor or targeting agent that works by lowering the amount of a splice variant of the androgen receptor in prostate cancer cells. The androgen receptor splice variant may be the V7 variant. The inhibitor or targeting agent may act to treat or prevent prostate cancer by lowering the amount of androgen receptor splice variant. As such the inhibitor may be effective in other proliferative diseases where splice variants occur.

In one embodiment, the targeting compound may be a compound which targets the JMJD6 gene and prevent or reduce expression of the JMJD6 gene. For example, the targeting agent may be a may be an antisense oligonucleotide or a mediator of RNA interference (RNAi) such as an siRNA (small interfering RNA), shRNA (short hairpin RNA).

RNAi is a biological pathway that can be used for regulation or inhibition of gene expression. Antisense oligonucleotides can also induce regulation or inhibition of gene expression. Both of these approaches affect gene expression by use of an oligonucleotide sequence that binds to the target RNA via Watson and Crick base pairing.

Short hairpin RNA is a type of RNA interference (RNAi) which can be used for regulation or inhibition of gene expression. shRNA molecules generally comprise a first sequence of about 19-22 nucleotides followed by a second sequence of about 19-22 nucleotides, wherein the first and second sequence are complementary and can form a duplex. The first sequence or second sequence may be complementary to a sequence in a target region or gene. The first and second sequence are linked by a further sequence of nucleotides which forms a loop structure when the first and second sequences form a duplex.

siRNA can be used to effect transient reduction or inhibition of gene expression.

The JMJD6 targeting agent may be capable of inhibiting expression of the JMJD6 gene. The JMJD6 targeting agent may be selected from an antisense oligonucleotide or an RNAi mediator such as an siRNA, shRNA. The RNAi mediator or the antisense oligonucleotide may comprise a sequence complementary to the sequence of the JMJD6 gene.

In an embodiment the JMJD6 targeting agent reduces the production of one or more androgen receptor splice variant, e.g. within prostate cancer cells. Androgen receptor splice variants may be identified as variants of the androgen receptor which are C-terminally truncated and/or lack the canonical ligand-binding domain. There are multiple different splice variants of the androgen receptor. The JMJD6 targeting agent may reduce production of androgen splice variants selected from one or more of AR-V1, AR-V2, AR-V3, AR-V4, AR-V5, AR-V6, AR-V7, AR-V8, AR-V9, AR-V10, AR-V11, AR-V12, AR-V13, AR-V14, AR-V15, AR-V16 AR-V18, AR-V23, AR8, ARQ640X, ARv5es, ARv56es, ARv7es, AR-45, AR-V567es. In a preferred embodiment the JMJD6 targeting agent reduces the production of the androgen receptor slice variant AR-V7.

In an embodiment the JMJD6 targeting agent results in the degradation of JMJD6 in cells, or reduced expression of JMJD6 in cells.

In an aspect the invention relates to a pharmaceutical composition comprising a JMJD6 targeting agent, e.g. for use in the treatment of prostate cancer.

In another aspect, the invention relates to a pharmaceutical composition, comprising an androgen therapy and a JMJD6 targeting agent.

The pharmaceutical composition may further comprise one or more additional active agents, a pharmaceutically acceptable carrier, diluent, excipient or adjuvant. The composition may comprise another agent, such as an anti-androgen therapy.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the single domain antibody of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

A drug delivery system (DDS) may be used to deliver the JMJD6 targeting agent or pharmaceutical composition. The DDS may comprise synthetic biodegradable polymers, hydrophobic materials such as α-hydroxy acids, such as polylactic-co-glycolic acid [PLGA]), and polyanhydrides. The DDS may comprise naturally occurring polymers, such as complex sugars, such as hyaluronan, chitosan [CHI] and inorganics such as hydroxyapatite. The DDS may comprise metal nanoparticles such as gold nanoparticles. It may also include prodrug forms, such as those targeting the inhibitor to prostate/prostate cancer cells.

The pharmaceutical composition of the invention can be in the form of a liquid, e.g., a solution, emulsion or suspension. The liquid compositions of the invention, whether they are solutions, suspensions or other like form, can also include one or more of the following: sterile diluents such as water, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. The composition can be enclosed in an ampoule, a disposable syringe or a multiple-dose vial made of glass, plastic or other material.

An intravenous formulation of the JMJD6 targeting agent or pharmaceutical composition of the invention may be in the form of a sterile injectable aqueous or non-aqueous (e.g. oleaginous) solution or suspension. The sterile injectable preparation may also be in a sterile injectable solution or suspension in a non-toxic parenterally-acceptable diluent or solvent, for example, a solution in 1,3-butanediol. Among the acceptable vehicles and solvents that may be employed are water, phosphate buffer solution, Ringer's solution and isotonic sodium chloride solution. In addition, sterile, fixed oils may be employed as a solvent or suspending medium. For this purpose, any bland fixed oil may be employed, including synthetic mono- or diglycerides. In addition, fatty acids such as oleic acid may be used in the preparation of the intravenous formulation of the invention.

The pharmaceutical composition can be prepared using methodology well known in the pharmaceutical art. For example, a composition intended to be administered by injection can be prepared by combining a vector of the present invention with water so as to form a solution. A surfactant can be added to facilitate the formation of a homogeneous solution or suspension.

The JMJD6 targeting agent or pharmaceutical composition may be administered by any suitable route. For example, delivery may be oral, topical, parenteral, sublingual, rectal, vaginal, ocular, intranasal, pulmonary, intradermal, intravitreal, intratumoral, intramuscular, intraperitoneal, intravenous, subcutaneous, intracerebral, transdermal, transmucosal, by inhalation, or topical, particularly to the ears, nose, eyes, or skin or by inhalation.

Parenteral administration includes, for example, intravenous, intramuscular, intraarterial, intraperitoneal, intranasal, rectal, intravesical, intradermal, topical or subcutaneous administration.

The skilled person will be aware of how to prepare a suitable formulation for these administration routes.

The pharmaceutical composition can be in the form of a liquid, e.g., a solution, syrup, solution, emulsion or suspension. The liquid can be useful for oral administration or for delivery by injection, infusion (e.g., IV infusion) or subcutaneously.

When intended for oral administration, the composition can be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by intravenous injection or infusion.

The amount of the drug, i.e. JMJD6 targeting agent described herein that is effective/active in the treatment of prostate cancer will depend on the nature of the disease or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disease, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of the drug by weight of the composition. When intended for oral administration, this amount can be varied to range from about 0.1% to about 80% by weight of the composition. Preferred oral compositions can comprise from about 4% to about 50% of the drug by weight of the composition.

Compositions can be prepared so that a parenteral dosage unit contains from about 0.01% to about 2% by weight of the single domain antibody of the present invention.

For administration by injection, the composition can comprise from about typically about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks.

In one aspect, the invention relates to a method of treating prostate cancer comprising administering a therapeutically effective amount of a JMJD6 targeting agent or a pharmaceutical composition comprising a JMJD6 targeting agent. The JMJD6 targeting agent is as described herein.

In one aspect, the invention relates to a method of treating or preventing endocrine resistance in prostate cancer, e.g. advanced prostate cancer, comprising administering a therapeutically effective amount of a JMJD6 targeting agent or a pharmaceutical composition comprising a JMJD6 targeting agent.

In one aspect, the invention relates to a JMJD6 targeting agent for the manufacture of a medicament for the treatment of prostate cancer.

As used herein, “treat”, “treating” or “treatment” means inhibiting or relieving a disease or disease. For example, treatment can include a postponement of development of the symptoms associated with a disease or disease, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., human patients, being treated. Many medical treatments are effective for some, but not all, patients that undergo the treatment.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

As used herein, the term “effective amount” means an amount of the targeting agent, that when administered alone or in combination with an additional therapeutic agent to a cell, tissue, or subject, is effective to achieve the desired therapeutic or prophylactic effect under the conditions of administration

Compounds described herein are useful in treating prostate cancer or a prostatic disorder. A prostatic disorder refers to any disease that afflicts the prostate gland in the male reproductive system. The prostate gland is dependent on the hormonal secretions of the testes. Expression of JMJD6 has been detected in other cancers, more specifically in the neovasculature associated with these cancers. A wide range of carcinomas, including conventional (clear cell) renal cell, transitional cell of the bladder, testicular-embryonal, neuroendocrine, colon, and breast, and the different types of malignancies were found consistently and strongly to express JMJD6 in their neovasculature. In an embodiment the prostate cancer is selected from; acinar adenocarcinoma, ductal adenocarcinoma, transitional cell carcinoma (urothelial carcinoma), squamous cell prostate cancer, small cell prostate cancer, large cell prostate cancer, mucinous adenocarcinoma, signet cell prostate cancer, basal cell prostate cancer, leiomyosarcoma, rhabdomyosarcoma.

In an embodiment, the prostate cancer may be endocrine-resistant prostate cancer, or castration-resistant prostate cancer. Thus, the invention is particularly useful in treating endocrine-resistant prostate cancer, or castration-resistant prostate cancer.

The upregulation of AR-V7 is linked to endocrine resistance in advance prostate cancer, as such in an embodiment the invention relates to a JMJD6 targeting agent for use in the treatment or prevention or diagnosis of endocrine resistance in prostate cancer.

The JMJD6 targeting agent may be used in combination with an anti-cancer therapy which may be an existing therapy or therapeutic agent. The JMJD6 targeting agent may be used in combination with a further anti-cancer therapy. The further anti-cancer therapy may be selected from radiotherapy, chemotherapy, surgery, immunotherapy, checkpoint inhibitors, hormone therapy. In particular, the further anti-cancer therapy may be selected from therapies commonly used in the treatment of prostate cancer for example; enzalutamide, abiraterone/abiraterone acetate, apalutamide radium-223, docetaxel, sipuleucel-T, cabazitaxel, mitoxantrone, bicalutamide, ketoconazole, and/or corticosteroids. The further anti-cancer therapy may be administered simultaneously, sequentially or separately, with the JMJD6 targeting compound. In one embodiment, the therapy is an anti-androgen therapy.

In a specific embodiment of the present invention, the composition is administered concurrently with a chemotherapeutic agent or with radiation therapy. In another specific embodiment, the chemotherapeutic agent or radiation therapy is administered prior or subsequent to administration of the composition of the present invention, preferably at least an hour, five hours, 12 hours, a day, a week, a month, more preferably several months (e. g. up to three months), prior or subsequent to administration of composition of the present invention.

In an embodiment, the invention also relates to a combination therapy wherein the JMJD6 targeting agent, for example enhances the efficacy of the existing anticancer therapy, comprising administration of the JMJD6 targeting compound or composition of the invention and an anti-cancer therapy. In one embodiment, the therapy is an anti-androgen therapy, such as abiraterone/abiraterone acetate, enzalutamide or apalutamide.

In one embodiment, the effect is synergistic. The anti-cancer therapy may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include checkpoint inhibitors, antineoplastic agents, immunogenic agents, attenuated cancerous cells, tumour antigens, antigen presenting cells such as dendritic cells pulsed with tumour-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumour-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy, cisplatin, gefitinib, paclitaxel, doxorubicin, epirubicin, capecitabine, carboplatin, cyclophosphamide, 5 fluorouracil. In one embodiment, the therapy is selected from enzalutamide, abiraterone, radium-223, docetaxel, sipuleucel-T, cabazitaxel, mitoxantrone, bicalutamide, ketoconazole, and/or corticosteroids. In one embodiment, the JMJD6 targeting agent or composition is used in combination with surgery. The JMJD6 targeting agent or composition of the invention may be administered at the same time or at a different time as the other therapy, e.g., simultaneously, separately or sequentially.

In specific embodiments, it may be desirable to administer the JMJD6 targeting agent or composition of the present invention locally to the area in need of treatment such at as the site of a tumour. In another embodiment it may be desirable to administer the JMJD6 targeting agent or composition by intravenous injection or infusion. The amount of the JMJD6 targeting agent of the present invention that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of the JMJD6 targeting agent according to the present invention such that a suitable dosage will be obtained. The correct dosage of the compounds will vary according to the particular formulation, the mode of administration, and its particular site, host and the disease being treated. Other factors like age, body weight, sex, diet, time of administration, rate of excretion, condition of the host, drug combinations, reaction sensitivities and severity of the disease shall be taken into account. Administration can be carried out continuously or periodically.

In an aspect the present invention relates to a kit comprising a JMJD6 targeting agent or a pharmaceutical composition comprising a JMJD6 targeting agent. and instructions for use.

The kit may comprise additional components such as further anti-cancer therapies as described herein, one or more additional active agents, a pharmaceutically acceptable carrier, diluent, excipient or adjuvant.

In an aspect the present invention relates to a method of diagnosing or prognosing prostate cancer, comprising
a. obtaining a biological sample,
b. determining the level of JMJD6 in the sample; e.g. the gene expression level wherein an increased level of JMJD6 compared to a reference sample indicates a poor prognosis.

The expression level of JMJD6 may be detected/determined using a technique selected from reverse transcriptase-polymerase chain reaction (RT-PCR) methods, quantitative real-time PCR (qPCR), microarray, RNA sequencing (RNA-Seq), next generation RNA sequencing (deep sequencing), gene expression analysis by massively parallel signature sequencing (MPSS), or transcriptomics, antibody based methods, or proteomics. The expression level of JMJD6 detected may be the nuclear expression level. The method may further comprise bringing the nucleic acid into contact with a polynucleotide probe or primer comprising a polynucleotide sequence capable of hybridising selectively to the nucleotide sequence set out in SEQ ID NO. 1 or 2.

In an embodiment the method relates to a method of diagnosing or prognosing acinar adenocarcinoma, ductal adenocarcinoma, transitional cell carcinoma (urothelial carcinoma), squamous cell prostate cancer, small cell prostate cancer, large cell prostate cancer, mucinous adenocarcinoma, signet cell prostate cancer, basal cell prostate cancer, leiomyosarcoma, rhabdomyosarcoma.

The reference sample may be obtained from a healthy individual who does not have prostate cancer.

In an aspect the invention relates to a method of inhibiting androgen receptor splicing/the formation of androgen receptor splice variants comprising contacting a cell with a JMJD6 targeting agent.

The cell may be contacted with the JMJD6 targeting agent in vitro, in vivo or ex vivo.

In another aspect, the invention relates to a method of monitoring the therapeutic efficacy of a prostate cancer therapy, comprising; determining the level; e.g. the expression level, of JMJD6 prior to administration of the therapy and determining the level of JMJD6 after administration of the therapy.

The level of JMJD6 may be determined in conjunction with the level of AR-V7. The level of JMJD6 may be determined at multiple time points after administration of a therapy. The level of JMJD6 determined may be the protein expression level or the gene expression level. The level of JMJD6 may be detected using a technique selected from reverse transcriptase-polymerase chain reaction (RT-PCR) methods, quantitative real-time PCR (qPCR), microarray, RNA sequencing (RNA-Seq), next generation RNA sequencing (deep sequencing), gene expression analysis by massively parallel signature sequencing (MPSS), or transcriptomics. The expression level of JMJD6 detected may be the nuclear expression level.

In a further aspect, we provide a method of inhibiting growth of tumor cells, in particular in the prostate, in a subject/treating a subject having prostate cancer, comprising administering to a subject a therapeutically effective amount of a JMJD6 targeting agent or pharmaceutical composition described herein.

In a further aspect, we provide a method for the treatment or prevention of endocrine resistance in prostate cancer comprising administering to a subject a therapeutically effective amount of a JMJD6 targeting agent or pharmaceutical composition described herein.

In an aspect, we provide a method of identifying a JMJD6 targeting agent, comprising contacting a cell with a test compound and determining the level of androgen receptor splicing. The method may further comprise comparing the level of an androgen receptor splice variant to the wildtype androgen receptor. The method may also comprise comparing the level of an androgen receptor splice variant in the test sample with the level of an androgen receptor splice variant in a reference sample. The reference sample may comprise a sample which has not been exposed or contacted with the test compound.

We also provide a compound obtained or obtainable by a method comprising contacting a cell with a test compound and determining the level of androgen receptor splicing.

The cell may be contacted with the test compound in vitro, in vivo or ex vivo. The test compound may be a potential JMJD6 targeting agent identified via methods such as inhibitor screens or in silico modelling.

The level of androgen receptor splicing may be identified using a technique selected from reverse transcriptase-polymerase chain reaction (RT-PCR) methods, quantitative real-time PCR (qPCR), microarray, one-step reverse transcription quantitative PCR, RNA sequencing (RNA-Seq), next generation RNA sequencing (deep sequencing), gene expression analysis by massively parallel signature sequencing (MPSS), or transcriptomics.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety. The invention is further described in the non-limiting examples.

EXAMPLES

### Materials and Methods

### Patients and Tissue Samples

All patients had mCRPC treated at the Royal Marsden Hospital (RMH) and provided written informed consent, being enrolled into protocols approved by the RMH ethics review committee (reference no. 04/Q0801/60). Patient clinical data were retrospectively collected from the RMH electronic patient record system.

ICR/RMH cohort. 74 previously collected biopsies were identified as having sufficient formalin-fixed, paraffin embedded (FFPE) mCRPC tissue for assessment (Bone, n=41; Lymph node, n=21; Liver, n=4; Other, n=8). Of these, 64 also had matched, same-patient diagnostic CSPC biopsies available. All biopsy blocks were freshly sectioned and only considered for immunohistochemistry analyses if adequate material was present (50 tumor cells). All CSPC biopsies demonstrated adenocarcinoma.

International Stand Up To Cancer/Prostate Cancer Foundation (SU2C/PCF) cohort. Due to the low level of AR-V7 expression at diagnosis of castration-sensitive PC [13], the bioinformatic analyses of patient sequencing data presented in this study were intentionally performed using publicly accessible data obtained from only mCRPC patients. Whole exome (n=231) and transcriptome (n=159) sequencing data from mCRPC patients generated by the SU2C/PCF Prostate Cancer Dream Team were downloaded and reanalyzed [2]

### Antibody Validation

Antibody specificity was determined by Western blot (WB) analyses comparing detection of JMJD6 protein levels in LNCaP95 whole cell lysates cultured with either non-targeting control siRNA or ON-TARGETplus pooled JMJD6 siRNA (Dharmacon; GE healthcare). AR-V7 antibody validation was performed as previously described [13].

### Immunohistochemistry (IHC)

JMJD6 IHC was performed using a mouse anti-JMJD6 antibody (Santa Cruz Biotechnology; sc-28348; 200 ug/ml stock). Antigen retrieval was achieved by microwaving slides in pH 6 Antigen Retrieval Buffer (HDS05-100; TCS Biosciences) for 18-min at 800 W prior to incubation with anti-JMJD6 antibody (1:50 dilution) for 1-hour at room temperature. The reaction was visualized using the EnVision system (K4061; DAKO). Antibody specificity was confirmed from LNCaP95 cell pellets following treatment with ON-TARGETplus pooled JMJD6 siRNA, compared to non-targeting control siRNA. AR-V7 IHC was performed as previously described [13]. JMJD6 and AR-V7 quantification was determined by a pathologist blinded to clinical data using the modified H score (HS) method [14]; [(% of weak staining)×1]+[(% of moderate staining)×2]+[(% of strong staining)×3], to determine overall percentage JMJD6 positivity across the stained tumor sample (range: 0 to 300).

### Cell Lines and Cultures

All cell lines were purchased from LGC Standards/ATCC unless otherwise specified and grown in recommended media at 37° C. in 5% CO₂. Short tandem repeat profiling was performed using the Cell Authentication Service by Eurofins Medigenomix to ensure the quality and integrity of the cell lines used. Cells lines were tested for mycoplasma after thawing, then regularly every 6-8 weeks during culture using the Venor®GeM Advance Mycoplasma Detection Kit (Minerva Biolabs). Early passages were thawed every 3-months (after approximately 15-20 passages). Small interfering RNA (siRNA): All siRNAs were ONTARGETplus pools (Dharmacon; GE heathcare), and used in combination with 0.4% RNAiMax transfection reagent (ThermoFisher Scientific) as per manufacturer's instructions. siRNA experiments were performed at 50 nM, unless otherwise specified, for 72-hours.

JMJD6 plasmid overexpression: Wild-type pcDNA3-JMJD6-WT (JMJD6^{WT}) and the catalytically inactive mutants pcDNA3-JMJD6-ASM2 (MUT1) and pcDNA3-JMJD6-BM1 (MUT2) JMJD6 expression constructs were kindly donated by Dr. A. Böttger [15, 16] and transfected into 22Rv1 and VCaP cell lines using Lipofectamine 3000 (Invitrogen, Carlsbad, CA). All treatments were performed using 1 g of total plasmid. For experiments requiring lower concentrations, the empty vector control plasmid (pcDNA3) was added to JMJD6^{WT}, MUT1 or MUT2, respectively, to make up the difference (e.g. JMJD6^{WT}+0.5 g JMJD6^{WT}+0.5 g empty vector control plasmid=1 g total plasmid input). All plasmid overexpression experiments were performed in 2 mls total volume.

Drugs: Enzalutamide was from Selleckchem (S1250). Dimethyl sulfoxide (DMSO) was from Fisher Scientific (BP231-1). 2,4-Pyridinedicarboxylic acid (2,4-PDCA) was purchased from Sigma-Aldrich (04473).

### Growth Assays

Cells were plated in 48-well tissue culture plates and treated as indicated the following day, then grown for 6-days or until 80-90% confluence. To quantify growth of LNCaP, LNCaP95, 22Rv1 and PNT2 cell lines, cells were fixed with 10% (w/v) aqueous trichloroacetic acid and incubated at 4° C. for 30-minutes prior to washing and air-drying. Subsequently cells were stained with sulforhodamine B (SRB) for 30-minutes prior to excess dye removal with 1% (v/v) aqueous acetic acid and further air-drying. Following this, protein-bound dye was dissolved in 10 mM Tris base solution, transferred to a 96-wellplate, and optical density determined at 510 nm using the Synergy HT microplate reader (BioTek). VCaP cell growth assays were analyzed using CellTiter-Glo® Luminescent Cell Viability Assay (Promega) as per manufacturer instructions, and luminescence quantified using the Synergy HT microplate reader (BioTek).

### Western Blot (WB)

Subsequent to cell lysis with RIPA buffer (Pierce) supplemented with cOmplete™ EDTA-free Protease Inhibitor Cocktail (Roche), protein extracts (20 μg) were separated by electrophoresis on 4-12% NuPAGE® Bis-Tris gel plates (Invitrogen) prior to transfer onto Immobilon-P™ PVDF membranes of

0.45 μm pore size (Millipore). Membranes were then incubated sequentially with primary and then secondary antibody in 5% milk and tris-buffered saline (TBS) and Tween®20 (Sigma Aldrich). Chemiluminescence was then detected using the Chemidoc Touch imaging system (BioRad).
Quantitative Reverse Transcription Polymerase Chain Reaction (qRT-PCR)

The RNeasy Plus Mini kit (Qiagen) was used to extract cellular RNA as per manufacturer's instructions. Following cDNA synthesis with the First Strand cDNA Synthesis Kit (Roche), qRT-PCR was performed using the ViiA™ 7 System Real-Time PCR System (Life Technologies) and the TaqMan Universal PCR Master Mix (Applied Biosystems) and probes (ThermoFisher Scientific) [11]. The fold change in mRNA expression levels was calculated by the comparative Ct method, using the formula 2-(-(ΔΔCt) [17].

### RNA Immunoprecipitation (RIP) Assays

Cells were transfected with either 25 nM non-targeting control siRNA (Dharmacon) or 25 nM JMJD6 siRNA (Dharmacon) using Lipofectamin RNAiMax (Invitrogen) and OPTI-MEM media (Gibco) as per manufacturer's instructions. After 72-hours, cells were cross-linked with 0.3% (v/v) aqueousformaldehyde (Thermo Scientific). RIP assays were performed using the EZ-Magna RIP (Cross-linked) Nuclear RNA-binding Protein Immunoprecipitation Kit (Millipore; 17-10521) following the manufacturer's protocol, and immunoprecipitated with 4 μg of U2AF65 antibody (Sigma Aldrich). RNA purification and DNAse I treatment was performed using RNeasy Plus Universal Mini Kit (Qiagen). The resultant RNAs were subjected to cDNA synthesis and RT-qPCR analysis. RIP data were derived from two independent experiments

### RNA-Seq and Analysis of Alternative Splicing Events

RNA-seq analysis comparing (1) LNCaP and LNCaP95 PC cells, and (2) LNCaP95 PC treated with either I-BET151 or vehicle (DMSO 0.1%), were performed as previously described [11]. Analyses compared impact of I-BET151 at concentrations of 500 nM and 2M for 8- and 48-hours which downregulate AR-V7 [11]) and equivalent vehicle (DMSO 0.1% for 8 and 48-hours). Only genes with baseline expression, as measured by Fragments Per Kilobase of transcript per Million mapped reads (FPKM), greater than the median expression level of all 315 spliceosome related genes at baseline across both experiments were included for analysis, with the top 15 genes most differentially expressed (FPKM) in each experiment (up- or down-regulated) being considered as genes of interest. For RNA-seq analyses of LNCaP95 PC cells treated with JMJD6 siRNA compared to non-targeting control siRNA, cellular RNA was extracted using the RNeasy Plus Mini Kit (Qiagen) as per manufacturer's instructions. RNA quality was analyzed using the Agilent RNA Screentape assay; 100 ng of total RNA from each sample was used for Agilent SureSelect library prep kit. Library quality was confirmed using the Agilent Bioanalyzer High Sensitivity DNA screentape Assay. The libraries were quantified and normalized by qPCR using Qiagen Generead Quantification Kit (Roche). Library clustering was performed on a cBot with Illumina HiSeq PE Cluster kit v3. The libraries were sequenced as paired-end 101-base-pair reads on an Illumina HiSeq 2500 membrane with an Illumina HiSeq SBS kit v3. Base-calling and quality scoring were performed using Real-Time Analysis (version 1.18.64) and FASTQ file generation and de-multiplexing using BCL2FASTQ. Paired end raw reads in FASTQ format were aligned to the reference human genome (hg19) using RNA-seq spliced read mapper TopHat (v2.0.7), with default settings [18]. The library and mapping quality were estimated using Picard tools (http://broadinstitute.github.io/picard).

Alternative splicing events (skipped exons, alternative 5′ splice sites, alternative 3′ splice sites, mutually exclusive exons and retained introns) based on Ensembl v61 annotation were accessed using MATS v3.0.8 [19].

### Spliceosome Related Gene Set

The list of genes relating to the spliceosome utilized to conduct this study was determined through interrogation and amalgamation of search results from two publicly accessible databases: 1) The Gene Ontology (GO) Resource [20-22]; search-term “spliceosome” with filters “Homo sapiens” and “UniProtKB”, and 2) The Molecular Signatures Database [23, 24]; search-term “SPLICING/SPLICEOSOME/SPLICEOSOMAL”.

### AR Activity, AR-V7 Activity, and Gene Expression Evaluation

Paired-end transcriptome sequencing reads were aligned to the human reference genome (GRCh37/hg19) using Tophat2 (v2.0.7). Gene expression levels, as measured by FPKM, were calculated using Cufflinks [26]. AR signaling activity was established by determining expression levels of either (1) 43-genes regulated by AR in PC cell line and metastatic prostate cancer RNA-seq datasets, as previously described (AR signature;), or (2) the HALLMARK_ADROGEN_RESPONSE gene set from the MSidDB (M5908 [25]; Androgen response (H);). AR-V7 signaling activity was determined using the previously published AR-V7-associated signature based on the expression levels of 59-genes associated with AR-V7 expression in mCRPC (AR-V7 signature; [13]).

Liquid Chromatography Mass Spectrometry (LC-MS) assays for JMJD6 inhibition by 2,4-PDCA Hydroxylation of a 12-mer peptide substrate (NPKRSRSREHRR, prepared with a C-terminal amide) of the LUC7L2 pre-mRNA splicing factor by JMJD6 (1-362, prepared as reported) [26, 27] was monitored by liquid chromatography mass spectrometry (LC-MS) using an Agilent 1290 infinity II LC system equipped with an Agilent 1290 infinity binary pump and coupled to an Agilent 6550 Accurate Mass Quadrupole Time of Flight (Q-TOF) mass spectrometer. Note this construct has hydroxylation but not demethylation activity [26]. All JMJD6₁₋₃₆₂ enzyme reactions were performed in 50 mM Tris.Cl pH 7.5 (prepared fresh each day) at 37° C. L (+)-Ascorbic acid sodium salt (code 11140), ferrous ammonium sulphate (FAS) as ammonium iron (II) sulphate hexahydrate (215406), and 2OG were from Sigma Aldrich (Poole, Dorset). The LUC7L2 peptide substrate was synthesized to >95% purity (LC-MS) by GL-Biochem (Shanghai, China). L-Ascorbic Acid (50 mM in deionized water), 2OG (10 mM in deionized water) and iron (II) sulphate (400 mM in 10 mM HCl) solutions were prepared freshly each day. JMJD6₁₋₃₆₂ (10 M) was pre-incubated with an 8-point and 3-fold serial dilution of 2,4-PDCA (100-0.046 M) for 15 minutes and the enzyme reaction initiated by addition of LUC7L2 substrate (100 M LUC7L2, 400 M L-ascorbate, 100 M FAS, 500 M 2OG final concentrations). The enzyme reaction was progressed for 2 hours at 37° C., then stopped by addition of formic acid to a final concentration of 1.0% (v/v). The quenched enzyme reaction was injected (6 l injections) onto a Proswift RP-4H 1×50 mm LC column (Thermo) and the LUC7L2 and LUC7L2-hydroxylated peptides were fractionated using a linear gradient of Solvent A (0.1% (v/v) formic acid in LCMS water) and Solvent B (0.1% (v/v) formic acid in 100% LCMS grade acetonitrile). Details of the gradient conditions, flow rates and maximum pressure limits are summarized in. Peptide ionization was monitored in the positive ion electrospray ionisation (ESI) mode with a drying gas temperature of 280° C., a drying gas flow rate of 13 L/minute, nebulizer gas pressure of 40 PSI, sheath gas temperature of 350° C., sheath gas flow rate of 12 L/min and a nozzle voltage of 1000V. Ion chromatogram data for the +2 charge state of both the non-hydroxylated and hydroxylated peptides were extracted and integrated using MassHunter qualitative software (Agilent). The % conversion of the peptide substrate to the +16 hydroxylated peptide was calculated using the equation: % conversion=100×hydroxylated/(hydroxylated+non-hydroxylated peptide). The IC₅₀ for 2,4-PDCA was determined from non-linear regression curve fitting using GraphPad prism 6.0.

### Statistical Analysis

All statistical analyses were performed using Stata v13.1 or GraphPad Prism v7 and are indicated within all figures and tables. Spearman's correlation was used to determine the association between JMJD6 and U2AF65 mRNA levels and other characteristics such as Androgen response (H), AR signature and AR-V7 signature. H-Scores are reported as median values and interquartile ranges. Comparison of JMJD6 expression levels between CSPC and mCRPC tissue samples, and correlations with next generation sequencing (NGS) data, were determined using the Wilcoxon matched-pair signed rank test. Comparisons between JMJD6 and AR-V7 expression levels in mCRPC tissue samples made using Mann-Whitney test. OS from CRPC biopsy was defined as time from CRPC biopsy to date of death. Survival analyses were estimated using the Kaplan-Meier method.

### Results

### Example 1. Orthogonal Analyses Identify the 2OG-Dependent Dioxygenase JMJD6 as a Regulator of AR-V7 Expression

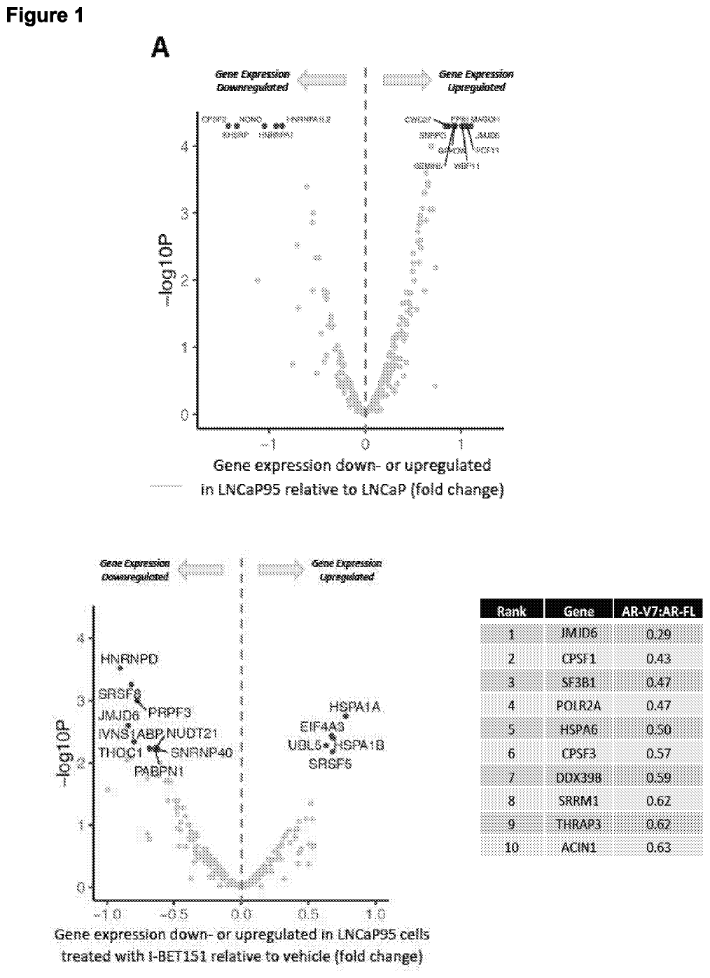

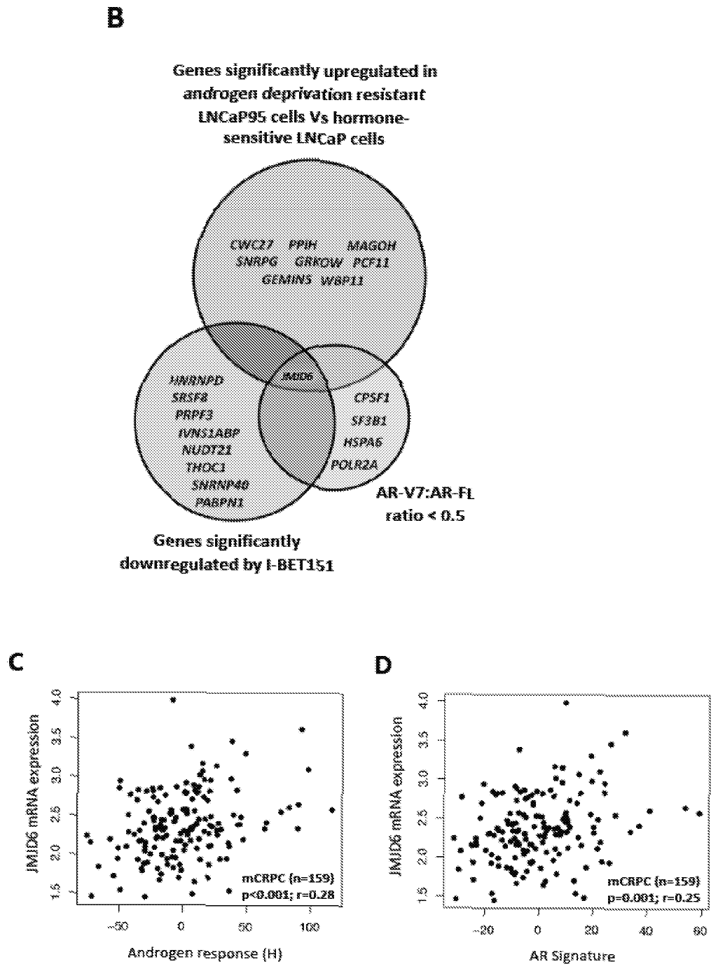

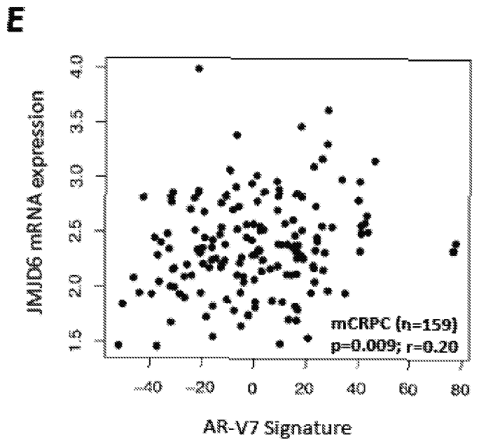

To identify proteins downregulated by BET inhibition that are critical to the regulation of AR-V7 splicing, an orthogonal three-stage investigative triangulation approach was employed (FIG. 1A). First, RNA-seq data from hormone-sensitive LNCaP cells (that do not produce AR-V7 protein) and their derivative, androgen deprivation resistant LNCaP95 cells (that do produce AR-V7 protein), were interrogated to identify which genes with roles relating to the spliceosome, as determined by GO annotations and The Molecular Signatures Database (Spliceosome related gene set), are significantly upregulated in LNCaP95 cells relative to LNCaP cells. These results were subsequently aligned with RNA-seq analyses comparing LNCaP95 PC cells treated with either a BET inhibitor (GSK1210151A; I-BET151) or vehicle (DMSO 0.1%), to investigate which of the spliceosome related gene set were also significantly downregulated by BET inhibition, which we, and others, have previously reported downregulates AR-V7 expression [11, 28]. To identify spliceosome related proteins that preferentially regulate AR-V7 generation, these transcriptomic data were amalgamated with the results of a targeted siRNA screen where all 315 genes in the spliceosome related gene set were individually silenced in the castration-resistant AR-V7 expressing PC cell lines LNCaP95 and 22Rv1 to determine their impact on AR-V7 protein levels relative to full-length AR (AR-FL) by WB. Genes were ranked in an order determined by the degree of AR-V7 downregulation relative to AR-FL averaged across both cell lines, with proteins causing the greatest reduction in AR-V7:AR-FL ratio being ranked highest. Only genes that were (1) significantly upregulated in LNCaP95 cells relative to LNCaP cells, (2) significantly downregulated following BET inhibition, and (3) associated with a >50% reduction in AR-V7 protein expression relative to AR-FL, were considered to be of further interest. Strikingly, these three independent lines of investigation identified the 2OG-dependent dioxygenase JMJD6 as the only gene to meet all three criteria, indicating that it may be an important regulator of AR-V7 protein expression (FIG. 1B).

To investigate the nature of the relationship between BET inhibition, JMJD6 and AR-V7, WB analyses were performed using LNCaP95 cells treated with I-BET151 for 48 hours. I-BET151 treatment led to a concurrent dose-dependent reduction in both JMJD6 and AR-V7 protein expression, with these both occurring to a similar extent, and at the same concentrations of I-BET151 (FIG. 1C).

Having identified JMJD6 as a protein of interest with respect to AR-V7 regulation in vitro, publicly accessible patient data repositories were then interrogated to establish its potential clinical relevance. Analysis of whole exome sequencing data from 231 mCRPC patient biopsies (SU2C/PCF) revealed JMJD6 genomic alterations in 47% (n=108/231) of evaluated samples, with these being predominatelygains (37%; n=86/231) or amplifications (8%; n=18/231). Importantly, analysis of available corresponding transcriptome data from these 231 mCRPC patient biopsies (n=108) found that JMJD6 gene gain/amplification correlated with an increase in JMJD6 mRNA expression compared to samples without JMJD6 copy number gain/amplification (p=0.02). Furthermore, when all available transcriptome sequencing data were evaluated (n=159; SU2C/PCF), JMJD6 mRNA expression levels correlated significantly with androgen response (H) (r=0.28, p<0.001), AR signature (r=0.25, p=0.001), and a previously reported AR-V7 signature (r=0.20, p=0.009), in mCRPC biopsies (FIG. 1C-E). Taken together, these results indicated that the JMJD6 gene is expressed in mCRPC and that its presence is associated with both AR and AR-V7 signaling activity, supporting further evaluation of JMJD6 as a gene of interest in mCRPC.

### Example 2. JMJD6 Correlates with AR-V7 Protein Levels and a Worse Prognosis in mCRPC

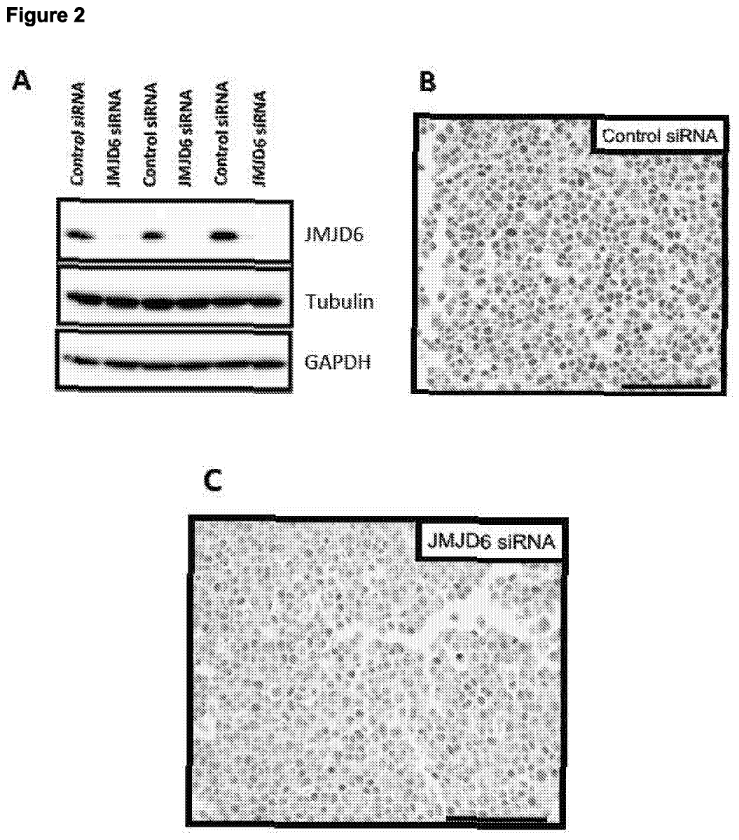

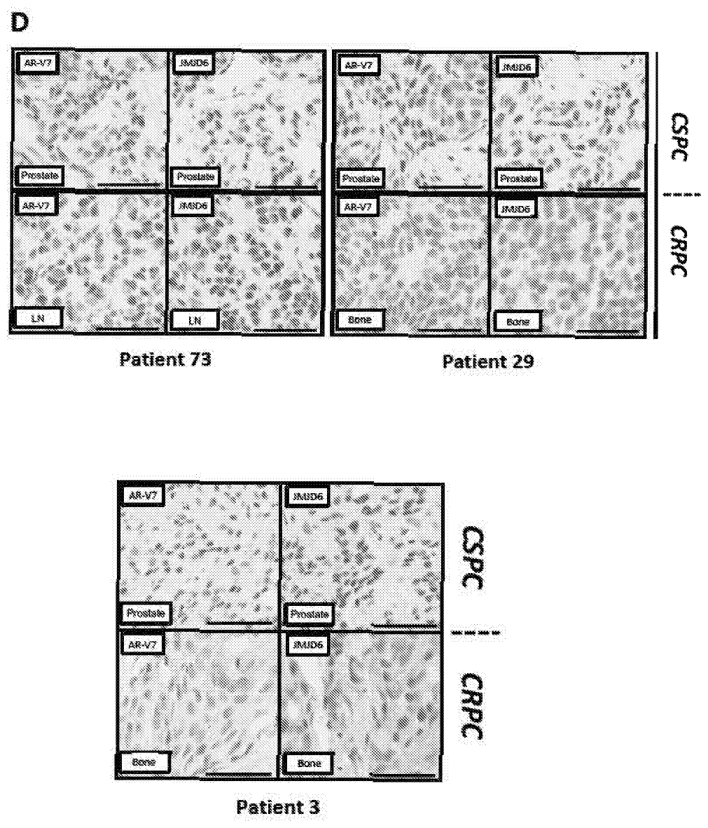

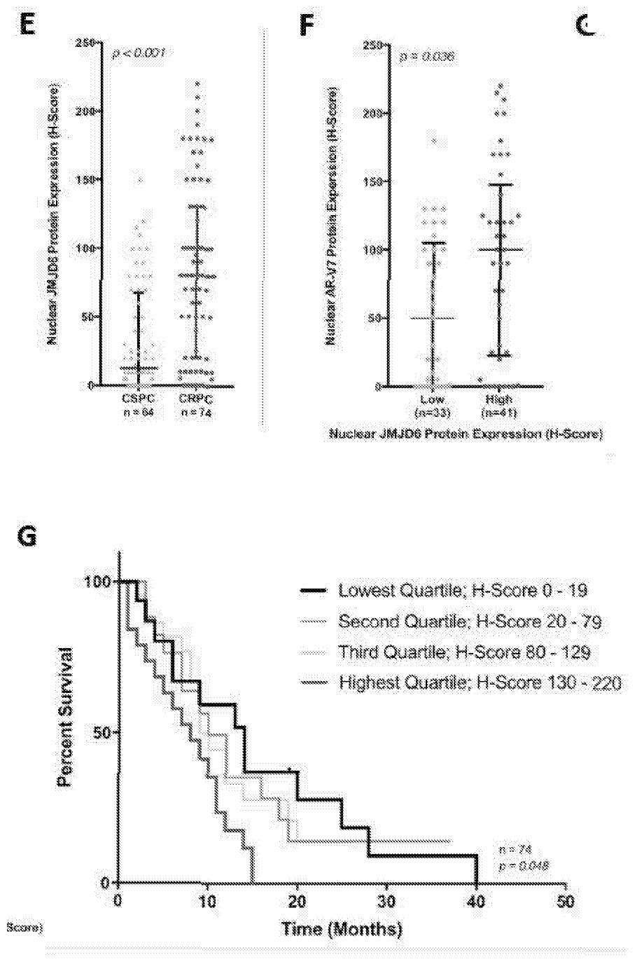

To further investigate the clinical significance of JMJD6 in lethal PC, we next validated an immunohistochemical assay for JMJD6 using whole cell lysates of LNCaP95 PC cells treated with either a non-targeting control siRNA or a JMJD6 specific siRNA (FIG. 2A-C), and then evaluated JMJD6 and AR-V7 protein levels in 74 mCRPC patient tissue biopsies (FIG. 2D;). Of these 74 patients, 64 patients also had sufficient matched, same patient, diagnostic, CSPC tissue available for analysis Nuclear JMJD6 protein expression increased significantly (p<0.001) as patients progressed from CSPC (median H-score 12.5, IQR [0.0-67.5]) to CRPC (80 [20.0-130.0]) (FIG. 2E). In addition, patients with higher nuclear JMJD6 expression (≥median H-score) had significantly (p=0.036) higher nuclear AR-V7 expression (100 [22.5-147.5]; n=41) than those patients with low nuclear JMJD6 expression (<median H-score; 50 [0.0-105.0]; n=33) (FIG. 2F). Finally, those patients with higher (≥75th percentile) nuclear JMJD6 expression had a significantly shorter survival than those patients with lower (<25th percentile) nuclear JMJD6 expression (14 months [n=16] vs 8 months [n=19]; hazard ratio 2.15; 95% confidence interval 1.19-5.92; p=0.017) (FIG. 2G).

Taken together, these data indicated that JMJD6 protein is produced in PC cells, that the level of JMJD6 increases significantly with the emergence of castration-resistant disease, and that this upregulation of JMJD6 correlates with a higher level of AR-V7. Whilst we appreciate that the heterogeneous nature and relatively limited size of the patient cohort presented makes definitive inferences on the impact of JMJD6 expression on survival challenging, in keeping with knowledge that AR-V7 expression is associated with a shorter OS, our results suggest that higher JMJD6 levels in mCRPC cells likely correlate with a worse prognosis. Overall, these data indicated that JMJD6 is a clinically relevant protein in mCRPC that merits further evaluation.

### Example 3 JMJD6 is Important for PC Cell Growth and Regulates AR-V7 Expression

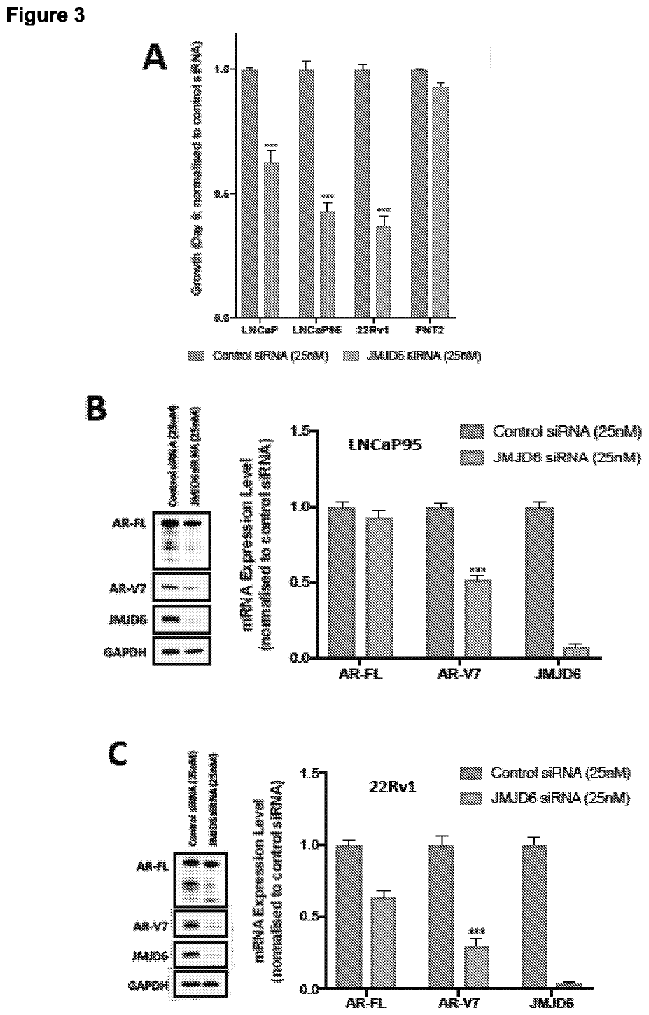

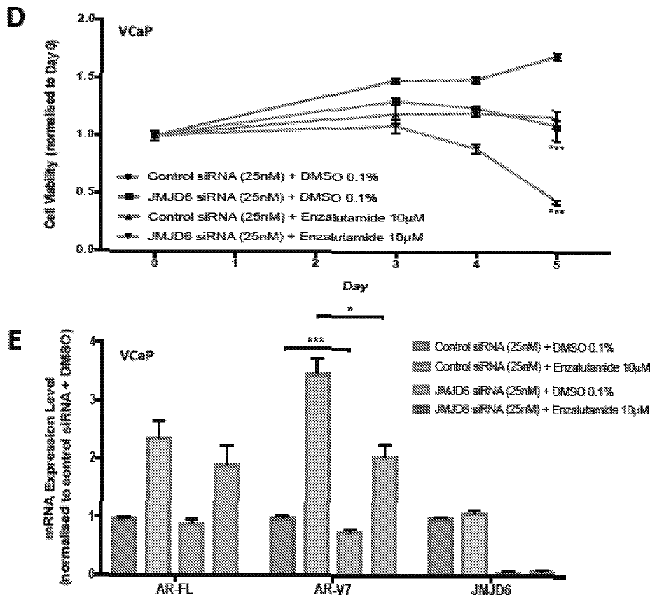

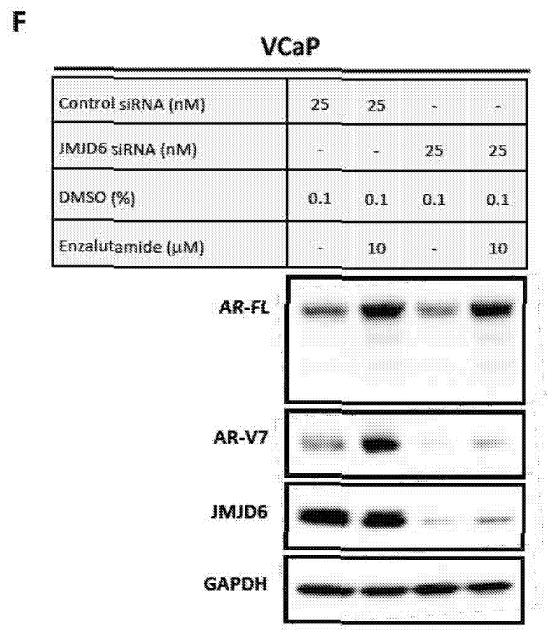

We next evaluated the impact of JMJD6 on PC cell growth and AR-V7 expression. Treatment with JMJD6 siRNA (25 nM) resulted in a significant reduction in the growth of the castration-resistant AR-V7-expressing PC cell lines LNCaP95 and 22Rv1, as evidence by a reduction in cell number, compared to treatment with non-targeting control siRNA (25 nM) (FIG. 3A). The growth of androgen-sensitive LNCaP cells was also significantly inhibited by JMJD6 siRNA knockdown. Interestingly however, the reduction in growth of LNCaP PC cells, which do not produce detectable levels of AR-V7 protein, was less than that seen with either its androgen-deprivation-resistant derivative LNCaP95, or 22Rv1 PC cells. PNT2 cells, which are an immortalized model of normal prostatic epithelium, were relatively unaffected. Notably, JMJD6 knockdown by siRNA (25 nM) for 72 hours downregulated both AR-V7 protein and mRNA levels (FIG. 3B-C). The effect of JMJD6 knockdown was also evaluated in the hormone-sensitive VCaP PC cell line, which contains the TMPRSS2/ERG rearrangement that is found in 30-40% of APCs, and which possesses a high copy gene amplification of AR. Furthermore, VCaP cells up-regulate the expression of AR-V7 in response to androgen-deprivation in vitro [29, 30]. VCaP Cells were treated with either a JMJD6 siRNA (25 nM) or a non-targeting control siRNA (25 nM), both with (Enzalutamide 10M) and without (DMSO 0.1%) AR blockade, and the effect on growth was determined after 5-days. As shown in FIG. 3D, JMJD6 siRNA knockdown reduced VCaP PC cell viability when compared to non-targeting control siRNA, as did treatment with enzalutamide alone. Importantly, however, combination treatment with JMJD6 siRNA and enzalutamide had a substantially more profound effect and inhibited VCaP cell viability more than either JMJD6 siRNA alone or enzalutamide treatment alone. To investigate this, RNA and WB analyses were performed using VCaP cells following 72 hour treatment with either non-targeting control siRNA or JMJD6 siRNA (25 nM), both with (Enzalutamide 10M) and without (DMSO 0.1%) AR blockade (FIG. 3E-F). JMJD6 knockdown downregulated AR-V7 RNA and protein levels, as previously observed in LNCaP95 and 22Rv1 cell lines (FIG. 3B-C); moreover, and critically, the upregulation of AR-V7 seen in response to AR blockade was also significantly attenuated by JMJD6 knockdown. Taken together, these data demonstrate that JMJD6 is important for PC cell viability and proliferation, and is required for the expression of AR-V7 in in vitro models of lethal PC.

### Example 4. JMJD6 Regulates AR-V7 Transcription in Part Through Recruitment of U2AF65 to AR-V7 Specific Splice Sites in In Vitro Models of CRPC

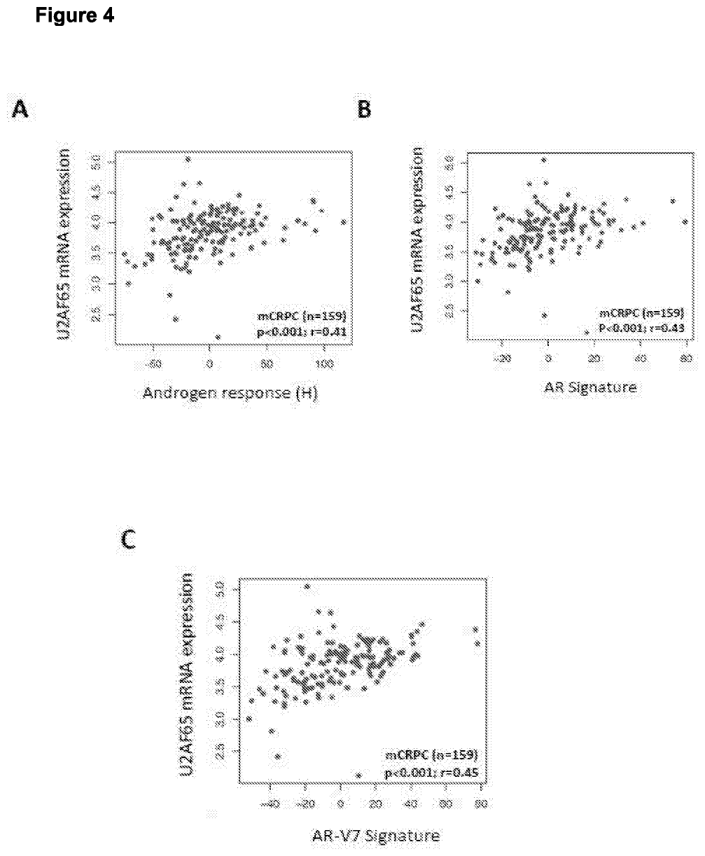

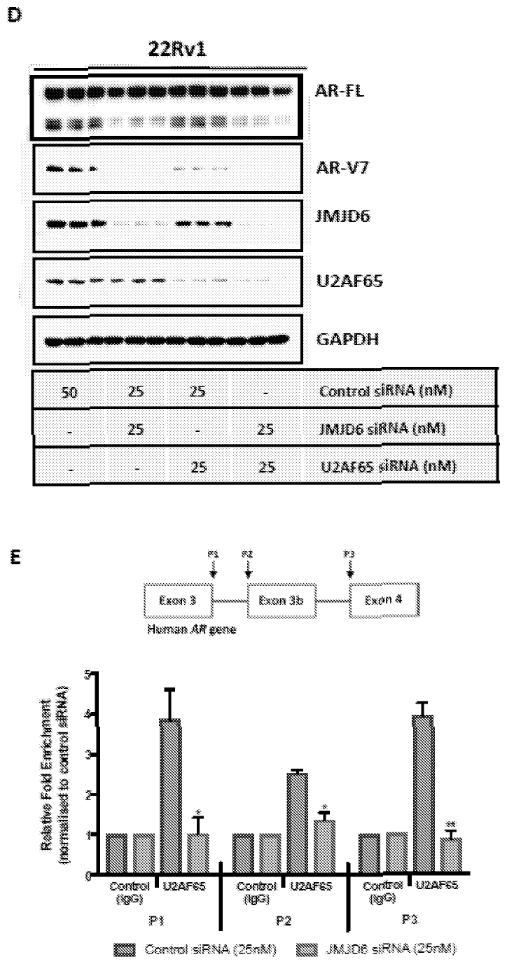

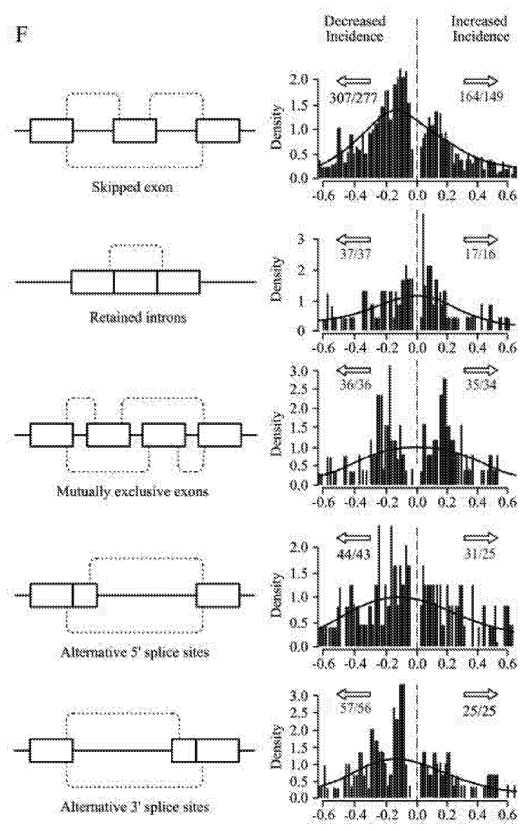

We next investigated the mechanism through which JMJD6 regulates AR-V7 production in preclinical models of CRPC. JMJD6 has been previously reported in the literature to interact with a number of proteins involved in RNA processing [15, 26, 27, 31]. Perhaps the best described example of this is its interaction with the splicing factor U2AF65, which has been demonstrated to be lysyl-5-hydroxylated by JMJD6 at residues in its arginine-serine rich region, including K15, K38 and K276 [27]. Importantly, U2AF65 is reported to play a critical role in the expression of AR-V7, having been shown to be recruited to AR-V7 specific splice sites in response to androgen deprivation therapy (ADT) [32]. Accordingly, as we observed for JMJD6, U2AF65 mRNA expression levels correlated significantly with androgen response (H) (r=0.41, p<0.001), AR signature (r=0.43, p<0.001), and AR-V7 signature (r=0.45, p<0.001) in mCRPC biopsies (FIG. 4A-C). We therefore hypothesized that JMJD6-mediated regulation of AR-V7 expression occurs through either the regulation of U2AF65 levels and/or its recruitment to AR-V7 specific splice sites. To determine the relationship between JMJD6, U2AF65 and AR-V7, we studied the impact of JMJD6 and U2AF65 protein depletion (both individually and concurrently) on the level of AR-V7, as well as on the levels of both JMJD6 and U2AF65 themselves, in 22Rv1 PC cells. JMJD6 siRNA (25 nM) and U2AF65 siRNA (25 nM) both decreased AR-V7 protein levels (FIG. 4D). JMJD6 siRNA had minimal impact on U2AF65 protein levels, and U2AF65 knockdown had no impact on JMJD6 expression, in keeping with reported data [31]. Having seen no effect of JMJD6 knockdown on U2AF65 expression, RIP analyses were performed to quantify the amount of U2AF65 bound to AR-V7 specific splice sites following JMJD6 siRNA knockdown (25 nM) compared to a non-targeting control siRNA, as per previously published protocols [32]. Antibodies against U2AF65, but not control IgG, precipitated AR pre-mRNA at the P1 (containing the 5′ splice site for both AR and AR-V7) and P2 (containing the 3′ splice site for AR-V7) regions in 22Rv1 cells treated with control siRNA; this effect being significantly reduced with JMJD6 siRNA (FIG. 4E). Taken together, these results indicate that JMJD6 regulates the recruitment of U2AF65 to AR-V7-specific splice sites.

To explore how JMJD6 regulates alternative splicing events in CRPC cells more broadly, RNA-seq analyses were performed of LNCaP95 PC cells prior to, and after, treatment with either JMJD6 siRNA or non-targeting control siRNA. Overall, JMJD6 knockdown led to substantial changes (determined by normalized-read count fold change >2 or <½ and false discovery rate <0.05) in 753 alternative splicing events involving 698 genes (FIG. 4F), with the majority of these occurring less frequently. Consistent with its assigned role in serine and arginine-rich (SR) protein modification and associated studies [26], these results indicate that JMJD6 knockdown reduces the overall incidence of alternative splicing events. Furthermore, in keeping with our previous result showing that JMJD6 knockdown downregulated AR-V7 expression (FIG. 3B-C and 3E-H), JMJD6 knockdown was found to reduce the mean AR-V7 signature score.

### Example 5. JMJD6-Mediated AR-V7 Generation is Dependent on JMJD6 Catalytic Activity, Which can be Chemically Inhibited to Downregulate AR-V7 Protein Expression

Having determined that JMJD6 regulates U2AF65 recruitment to AR-V7-specific splice sites, and given that JMJD6 has been previously demonstrated to hydroxylate U2AF65 [27], we next

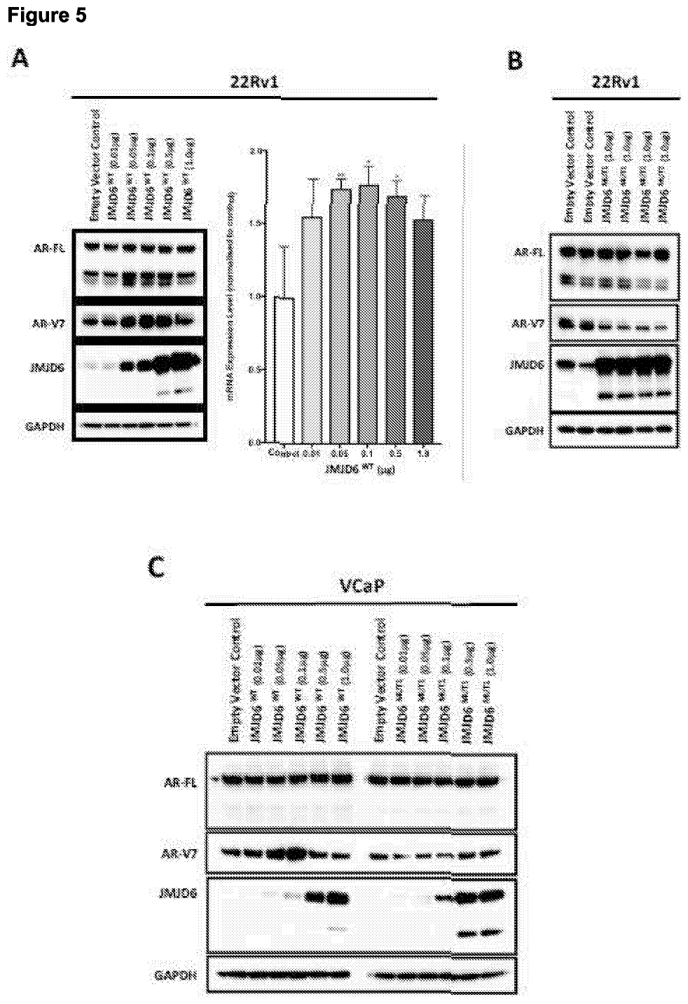

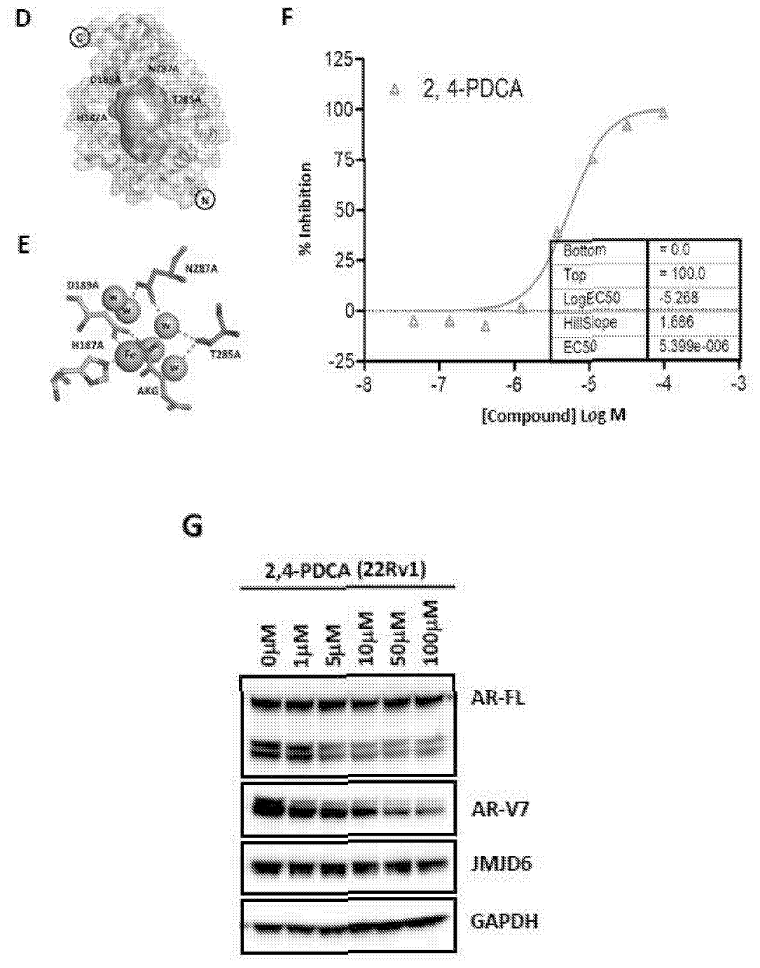

investigated the importance of a functional JMJD6 active site on AR-V7 levels. 22Rv1 PC cells were transfected with a JMJD6 wild-type (WT) plasmid (JMJD6^{WT}) for 72 hours; WB and RNA analyses demonstrated increased expression of both AR-V7 protein and mRNA with JMJD6 overexpression (FIG. 5A). Conversely, transfection with inactivating mutations of active site residues in the JMJD6 catalytic domain by pcDNA3-JMJD6-ASM2 (MUT1; D189A and H187A) [16] and pcDNA3-JMJD6-BM1 (MUT2; N287A and T285A) markedly decreased AR-V7 protein levels (FIG. 5B). To validate these findings, both JMJD6 WT , and the catalytically inactive mutant JMJD6^{MUT1}, were next transfected into the VCaP PC cell line; AR-V7 expression was induced by JMJD6^{WT}, but not by JMJD6^{MUT1} (FIG. 5C). Taken together, these results support the hypothesis that JMJD6-mediated increased expression of AR-V7 requires JMJD6 catalytic activity. Interestingly, the extent of AR-V7 upregulation in both 22Rv1 and VCaP PC cell lines was greater following transfection of lower concentrations of JMJD6^{WT} compared to higher concentrations of JMJD6^{WT}.

Importantly, studies of the physicochemical and geometric properties of JMJD6 with known drug targets such as protein kinases, interrogated using the canSAR drug discovery platform [33, 34], indicated that JMJD6 contains a ‘druggable’ pocket within its tertiary structure (defined as sites that harbor physiochemical and geometric properties consistent with binding orally-bioavailable small molecules [34]; (FIG. 5D-E;). Analogous pockets have been targeted in other 2OG oxygenases, in some cases leading to clinically approved drugs [35, 36]. Furthermore, consistent with crystallographic studies of JMJD6 [36, 37], these analyses demonstrated that the amino acids D189, H187A, N287 and T285, important for JMJD6 catalytic activity, lie within this druggable cavity.

To identify small molecule inhibitors of JMJD6 that would not disrupt its active site, liquid chromatography-mass spectrometry (LC-MS) analyses were performed. These identified the 2OG mimic pyridine-2,4-dicarboxylic acid (2,4-PDCA) as a JMJD6 inhibitor; 2,4-PDCA is a broad-spectrum, active site binding, 2OG competitive 2OG-dependent oxygenase inhibitor [35, 38, 39]. 2,4-PDCA caused dose-dependent reduction in isolated JMJD6-mediated lysyl-5-hydroxylation of the known downstream target LUC7-Like (LUC7L) [15, 26] (FIG. 5F). Having confirmed 2,4-PDCA to be an inhibitor of JMJD6 lysyl hydroxylase catalytic activity, we subsequently treated 22Rv1 PC cells with 2,4-PDCA for 48 hours. As shown in FIG. 5G, 2,4-PDCA resulted in a dose-dependent reduction in AR-V7 protein levels, supporting our previous siRNA and mutagenesis experiments. Taken together, these results support the proposal that a functional JMJD6-active site is required for AR-V7 protein production, and show that the JMJD6 active site is druggable. Thus, JMJD6 is a viable therapeutic target for drug discovery efforts to abrogate oncogenic AR-V7 signaling.

### Significance of Examples

Resistance to PC endocrine therapies including abiraterone and enzalutamide is inevitable and invariably fatal, and at least in part driven by constitutively active AR-SVs that remain undruggable. We have found that the 2OG-dependent dioxygenase JMJD6, which associates with worse prognosis and disease aggressiveness in multiple tumor types [40-43], plays an important role in PC biology, including in AR-V7 production. JMJD6 is expressed in PC and increases significantly with castration-resistance, with this increase associating with AR-V7 protein over-production in mCRPC biopsies and poorer survival. Our orthogonal investigations reveal JMJD6 to be critical for PC growth and a key regulator of AR-V7 expression. JMJD6 knockdown inhibits the upregulation of AR-V7 protein in response to AR blockade in hormone-sensitive VCaP PC cells. This is of therapeutic importance because for AR-V7 targeting to be successful, novel therapies are needed that can block AR-V7 generation rather than just counteract its oncogenic effects once EnR is established [13]. Moreover, the reduction in AR-V7 levels and PC cell growth seen following JMJD6 siRNA knockdown suggests limited functional redundancy, which is striking given that recently two other 2OG-dependent JmjC-domain containing oxygenases, JMJD1A/KDM3A and KDM4B [45], have also been reported to regulate AR-V7 generation. However, while JMJD1A/KDM3A and KDM4B are assigned as N-methyl lysine demethylases [46, 47], like other JmjC KDMs, other roles for them including N-methyl arginine demethylation are possible [48]. Given their roles in histone modification it is thus unclear as to what extent KDM4B/JMJD1A directly regulate AR splicing. Therefore, although it is likely that other 2OG-dependent JmjC-domain containing proteins play a role in the overall activity of the spliceosome machinery and AR splicing, albeit probably through alternative mechanisms, our results demonstrate that targeting the 2OG-dependent catalytic activity of JMJD6 is a promising PC drug discovery strategy. A better understanding of the interplay between these different proteins and the spliceosome machinery is now required.

The results indicate that JMJD6 regulates the expression of AR-V7, at least in part, by modulating the recruitment of the splicing factor U2AF65 to AR-V7 specific pre-mRNA splice sites, which we have previously shown to be critical for the expression of AR-V7 [32]. Moreover, our evidence implies the JMJD6-mediated regulation of AR-V7 expression is dependent on an intact JMJD6 catalytic site, which is in keeping with previous reports that JMJD6 lysyl-5-hydroxylates U2AF65 [27], and in doing so regulates U2AF65-mediated alternative splicing events [31]. Interestingly, however, the degree of AR-V7 upregulation in our studies was greater following transfection of lower concentrations of JMJD6^{WT} compared to higher concentrations of JMJD6^{WT}. This observation is consistent with, though does not prove, a role for JMJD6 induced catalysis in the increased production of AR-V7, as opposed to this occurring through a protein scaffold function of JMJD6 which would be expected to increase AR-V7 levels in line with JMJD6 levels. Given that JMJD6 is an Fe(II) and 2OG-dependent oxygenase, it is possible that the apparent dip in AR-V7 production despite the higher levels of JMJD6 reflects an inability of the cell to maintain optimal JMJD6 activity when it is overexpressed beyond a certain point due to a lack of Fe(II) and/or 2OG/dioxygen.

Importantly, our analyses reveal that the JMJD6 catalytic site resides within a druggable pocket, and we demonstrate that the known 2OG oxygenase inhibitor 2,4-PDCA, which we show to inhibit JMJD6 lysyl-5-hydroxylation, downregulates AR-V7 protein levels in castration-resistant PC cells. Taken together these findings point to a JMJD6/U2AF65/AR-V7 regulatory pathway, wherein JMJD6 enzymatic activity, most likely through hydroxylation of U2AF65, and/or other SR proteins, regulates U2AF65 recruitment to AR-V7 specific splice sites, which then facilitates the generation of AR-V7 through interaction with the spliceosome. Given that JMJD6 has the potential to hydroxylate/interact with SR proteins other than U2AF65 [15, 27, 49, 50], and may have other cellular functions, its biological roles are likely widespread and context-dependent. However, in light of the crucial role of AR-SVs, especially AR-V7 in CRPC, therapeutic modulation of its spliceosome regulatory roles may be particularly suited to PC treatment. Our demonstration that inhibition of JMJD6 by a broad-spectrum 2OG oxygenase inhibitor (that is an active site binding 2OG competitor) downregulates AR-V7 levels should promote the pursuit of more potent and selective JMJD6 inhibitors in future drug discovery efforts.

This is particularly relevant given the apparent pleiotropic roles of JMJD6 [49, 51]. However, 2OG oxygenases, are validated therapeutic targets, as shown by the clinical approvals of HIF prolyl hydroxylase inhibitors, which are active site Fe binding 2OG competitors [52].

Aside from the likelihood of its multiple context dependent substrates and partners [15, 26], the activity of JMJD6 could be limited by (local or global) iron, dioxygen or 2OG availability, as is the case for some, but not all, 2OG oxygenases including the hypoxia inducible factor prolyl hydroxylases [53]. 2OG is a vital intermediate in the TCA cycle and is generated by processes such as glutaminolysis. 2OG levels vary depending on cell replication rate, hypoxia, androgen deprivation, and genomic aberrations (e.g. PTEN loss) common in PC [2], therefore it is possible that variations in 2OG levels impact JMJD6 activity and hence AR-V7 levels.

Despite animal work demonstrating the importance of JMJD6 in development [49, 50], and extensive cellular studies, the lack of a validated downstream in vitro ‘read-out’ of physiologically relevant effects of JMJD6 catalysis is a significant obstacle in JMJD6 research. The effects of JMJD6 on AR-V7 levels are thus of general interest with respect to the role of JMJD6 in splicing and provide a means to study JMJD6 modulators, including inhibitors in cells. However, whilst the plasmids and methods used herein have been previously characterized [16, 27], without an established, quantifiable marker of JMJD6 catalysis in our models other than AR-V7, it is not possible to definitively state that the changes on AR-V7 levels observed are dependent solely on catalysis by JMJD6. This is of particular relevance when considering our overexpression and mutagenesis experiments; we are unable to ascertain the level of functionality of the expressed JMJD6^{WT}, nor that our mutants are completely inactive in cells where endogenous JMJD6 is present; this limits the strength with which inferences can be made on the importance of JMJD6 catalytic activity for AR-V7 expression. Thus, we cannot rule out that JMJD6-mediated regulation of AR-V7 involves a stoichiometric protein scaffold type interaction, which may or may not be linked to lysine-hydroxylation (or other JMJD6 catalyzed reaction). Indeed, a stoichiometric mechanism has been proposed for the AT hook domain of JMJD6 with respect to its role in adipogenesis in a manner independent of catalysis [54]. However, it should be noted that such a stoichiometric mechanism will be amenable to modulation of JMJD6 by binding of a therapeutic molecule, including but not limited to active site binding inhibitors.

To investigate the role of JMJD6 catalysis in regulating AR-V7 levels, we employed inhibition of JMJD6 with the small molecule 2,4-PDCA, which we found inhibits JMJD6 lysyl-5-hydroxylation and downregulates AR-V7 levels, supporting the proposal that catalysis by JMJD6 is implicated in AR-V7 upregulation. However, we employed 2,4-PDCA to provide ‘proof-of-principle’ evidence that PC cell inhibition of JMJD6 by an active site binding inhibitor is possible and impacts on AR-V7 protein levels. We appreciate that 2,4-PDCA is not optimised for therapeutic use and that such optimisation has been reported for inhibitors of other 2OG oxygenases, e.g. the hypoxia inducible factor prolyl hydroxylases. Thus, at least in some cell types, the permeability of 2,4-PDCA is low, with high concentrations being required to elicit its effects in vitro [55, 56]. 2,4-PDCA itself is thus unlikely to be useful for in vivo studies. Furthermore, 2,4-PDCA is a broad-spectrum2OG dioxygenase inhibitor and may inhibit other 2OG oxygenases, including JmjC-domain containing proteins. Selectivity can be achieved (and potency increased) by screening JMJD6 inhibitors against other 2OG oxygenases coupled with variation of non-optimal inhibitor compounds by structure activity relationship studies. In conclusion, through orthogonal analyses we identify JMJD6 as being critical to PC cell growth and an important regulator of AR-V7 protein levels in preclinical models of CRPC. Furthermore, JMJD6 inhibition has potential to overcome oncogenic AR-V7 signaling, and is an eminently tractable new therapeutic target for mCRPC that merits further evaluation in in vivo studies.

### REFERENCES

1. Visakorpi, T., et al., In vivo amplification of the androgen receptor gene and progression of human prostate cancer. Nat Genet, 1995. 9(4): p. 401-6.
2. Robinson, D., et al., Integrative clinical genomics of advanced prostate cancer. Cell, 2015. 161(5): p. 1215-1228.
3. de Bono, J. S., et al., Abiraterone and increased survival in metastatic prostate cancer. N Engl J Med, 2011. 364(21): p. 1995-2005.
4. Scher, H. I., et al., Increased survival with enzalutamide in prostate cancer after chemotherapy. N Engl J Med, 2012. 367(13): p. 1187-97.
5. Fizazi, K., et al., Abiraterone plus Prednisone in Metastatic, Castration-Sensitive Prostate Cancer. N Engl J Med, 2017. 377(4): p. 352-360.
6. Scher, H. I. and C. L. Sawyers, Biology of Progressive, Castration-Resistant Prostate Cancer: Directed Therapies Targeting the Androgen-Receptor Signaling Axis. Journal of Clinical Oncology, 2005. 23(32): p. 8253-8261.
7. Dehm, S. M., et al., Splicing of a novel androgen receptor exon generates a constitutively active androgen receptor that mediates prostate cancer therapy resistance. Cancer Res, 2008. 68(13): p. 5469-77.

8. Antonarakis, E., et al., Androgen receptor variant-driven prostate cancer: clinical implications and therapeutic targeting. Prostate Cancer Prostatic Dis, 2016. 19(3): p. 231-41.
9. Paschalis, A., et al., Alternative splicing in prostate cancer. Nat Rev Clin Oncol, 2018.
10. Antonarakis, E. S., et al., Clinical Significance of Androgen Receptor Splice Variant-7 mRNA Detection in Circulating Tumor Cells of Men With Metastatic Castration-Resistant Prostate Cancer Treated With First- and Second-Line Abiraterone and Enzalutamide. J Clin Oncol, 2017. 35(19): p. 2149-2156.
11. Welti, J., et al., Targeting bromodomain and extra-terminal (BET) family proteins in castration resistant prostate cancer (CRPC). Clin Cancer Res, 2018.
12. Pervaiz, M., P. Mishra, and S. Gunther, Bromodomain Drug Discovery—the Past, the Present, and the Future. Chem Rec, 2018. 18(12): p. 1808-1817.
13. Sharp, A., et al., Androgen receptor splice variant-7 expression emerges with castration resistance in prostate cancer. J Clin Invest, 2019. 129(1): p. 192-208.
14. Detre, S., G. Saclani Jotti, and M. Dowsett, A “quickscore” method for immunohistochemical semiquantitation: validation for oestrogen receptor in breast carcinomas. J Clin Pathol, 1995. 48(9): p. 876-8.
15. Heim, A., et al., Jumonji domain containing protein 6 (Jmjd6) modulates splicing and specifically interacts with arginine-serine-rich (RS) domains of SR- and SR-like proteins. Nucleic Acids Res, 2014. 42(12): p. 7833-50.
16. Mantri, M., et al., Crystal structure of the 2-oxoglutarate- and Fe(II)-dependent lysyl hydroxylase JMJD6. J Mol Biol, 2010. 401(2): p. 211-22.
17. Winer, J., et al., Development and validation of real-time quantitative reverse transcriptase-polymerase chain reaction for monitoring gene expression in cardiac myocytes in vitro. Anal Biochem, 1999. 270(1): p. 41-9.
18. Kim, D. and S. L. Salzberg, TopHat-Fusion: an algorithm for discovery of novel fusion transcripts. Genome Biol, 2011. 12(8): p. R72.
19. Shen, S., et al., MATS: a Bayesian framework for flexible detection of differential alternative splicing from RNA-Seq data. Nucleic Acids Res, 2012. 40(8): p. e61.
20. Ashburner, M., et al., Gene ontology: tool for the unification of biology. The Gene Ontology Consortium. Nature genetics, 2000. 25(1): p. 25-29.
21. Abida, W., et al., Genomic correlates of clinical outcome in advanced prostate cancer. Proc Natl Acad Sci U S A, 2019. 116(23): p. 11428-11436.
22. Carbon, S., et al., AmiGO: online access to ontology and annotation data. Bioinformatics, 2009. 25(2): p. 288-9.
23. Subramanian, A., et al., Gene set enrichment analysis: A knowledge-based approach for interpreting genome-wide expression profiles. Proceedings of the National Academy of Sciences, 2005. 102(43): p. 15545.
24. Liberzon, A., et al., Molecular signatures database (MSigDB) 3.0. Bioinformatics, 2011. 27(12): p. 1739-40.
25. Liberzon, A., et al., The Molecular Signatures Database (MSigDB) hallmark gene set collection. Cell Syst, 2015. 1(6): p. 417-425.
26. Islam, M. S., et al., Biochemical and structural investigations clarify the substrate selectivity of the 2-oxoglutarate oxygenase JMJD6. J Biol Chem, 2019. 294(30): p. 11637-11652.
27. Webby, C. J., et al., Jmjd6 catalyses lysyl-hydroxylation of U2AF65, a protein associated with RNA splicing. Science, 2009. 325(5936): p. 90-3.
28. Asangani, I. A., et al., BET Bromodomain Inhibitors Enhance Efficacy and Disrupt Resistance to AR Antagonists in the Treatment of Prostate Cancer. Mol Cancer Res, 2016. 14(4): p. 324-31.
29. van Bokhoven, A., et al., Molecular characterization of human prostate carcinoma cell lines. Prostate, 2003. 57(3): p. 205-25.
30. Yu, Z., et al., Rapid induction of androgen receptor splice variants by androgen deprivation in prostate cancer. Clin Cancer Res, 2014. 20(6): p. 1590-600.
31. Yi, J., et al., JMJD6 and U2AF65 co-regulate alternative splicing in both JMJD6 enzymatic activity dependent and independent manner. Nucleic Acids Res, 2017.
45(6): p. 3503-3518.
32. Liu, L. L., et al., Mechanisms of the androgen receptor splicing in prostate cancer cells. Oncogene, 2014. 33(24): p. 3140-50.
33. Bulusu, K. C., et al., canSAR: updated cancer research and drug discovery knowledgebase. Nucleic Acids Res, 2014. 42(Database issue): p. D1040-7.
34. Tym, J. E., et al., canSAR: an updated cancer research and drug discovery knowledgebase. Nucleic Acids Res, 2016. 44(D1): p. D938-43.
35. Rose, N. R., et al., Inhibition of 2-oxoglutarate dependent oxygenases. Chem Soc Rev, 2011. 40(8): p. 4364-97.
36. Yeh, T. L., et al., Molecular and cellular mechanisms of HIF prolyl hydroxylase inhibitors in clinical trials. Chem Sci, 2017. 8(11): p. 7651-7668.
37. Leung, I. K., et al., Structural and mechanistic studies on gamma-butyrobetaine hydroxylase. Chem Biol, 2010. 17(12): p. 1316-24.
38. Bonnici, J., et al., Inhibitors of both the N-methyl lysyl- and arginyl-demethylase activities of the JmjC oxygenases. Philos Trans R Soc Lond B Biol Sci, 2018.
373(1748).
39. Thalhammer, A., et al., Inhibition of the histone demethylase JMJD2E by 3-substituted pyridine 2,4-dicarboxylates. Organic & Biomolecular Chemistry, 2011.
9(1): p. 127-135.
40. Aprelikova, O., et al., The epigenetic modifier JMJD6 is amplified in mammary tumors and cooperates with c-Myc to enhance cellular transformation, tumor progression, and metastasis. Clin Epigenetics, 2016. 8: p. 38.
41. Wang, F., et al., JMJD6 promotes colon carcinogenesis through negative regulation of p53 by hydroxylation. PLoS Biol, 2014. 12(3): p. e1001819.
42. Zhang, J., et al., High expression of JMJD6 predicts unfavorable survival in lung adenocarcinoma. Tumour Biol, 2013. 34(4): p. 2397-401.
43. Lee, Y. F., et al., JMJD6 is a driver of cellular proliferation and motility and a marker of poor prognosis in breast cancer. Breast Cancer Res, 2012. 14(3): p. R85.
44. Fan, L., et al., Histone demethylase JMJD1A promotes alternative splicing of AR variant 7 (AR-V7) in prostate cancer cells. Proc Natl Acad Sci U S A, 2018. 115(20): p. E4584-e4593.
45. Duan, L., et al., Histone lysine demethylase KDM4B regulates the alternative splicing of the androgen receptor in response to androgen deprivation. Nucleic Acids Res, 2019. 47(22): p. 11623-11636.
46. Wilson, C. and A. J. Krieg, KDM4B: A Nail for Every Hammer? Genes (Basel), 2019. 10(2).
47. Yamane, K., et al., JHDM2A, a JmjC-containing H3K9 demethylase, facilitates transcription activation by androgen receptor. Cell, 2006. 125(3): p. 483-95.
48. Walport, L. J., et al., Arginine demethylation is catalysed by a subset of JmjC histone lysine demethylases. Nat Commun, 2016. 7: p. 11974.
49. Bottger, A., et al., The oxygenase Jmjd6—a case study in conflicting assignments. Biochem J, 2015. 468(2): p. 191-202.
50. Kwok, J., et al., Jmjd6, a JmjC Dioxygenase with Many Interaction Partners and Pleiotropic Functions. Front Genet, 2017. 8: p. 32.
51. Kwok, J., et al., Jmjd6, a JmjC Dioxygenase with Many Interaction Partners and Pleiotropic Functions. Front Genet, 2017. 8.
52. Chen, R. and N. Forsyth, Editorial: The Development of New Classes of Hypoxia Mimetic Agents for Clinical Use. Frontiers in Cell and Developmental Biology, 2019.
7: p. 120.
53. Mahon, P. C., K. Hirota, and G. L. Semenza, FIH-1: a novel protein that interacts with HIF-1alpha and VHL to mediate repression of HIF-1 transcriptional activity. Genes Dev, 2001. 15(20): p. 2675-86.
54. Reyes-Gutierrez, P., J. W. Carrasquillo-Rodriguez, and A. N. Imbalzano, Promotion of adipogenesis by JMJD6 requires the AT hook-like domain and is independent of its catalytic function. PLoS One, 2019. 14(8): p. e0216015.
55. Tschank, G., et al., Pyridinedicarboxylates, the first mechanism-derived inhibitors for prolyl 4-hydroxylase, selectively suppress cellular hydroxyprolyl biosynthesis. Decrease in interstitial collagen and Clq secretion in cell culture. Biochem J, 1987. 248(3): p. 625-33.
56. Kristensen, L. H., et al., Studies of H3K4me3 demethylation by KDM5B/Jarid1B/PLU1 reveals strong substrate recognition in vitro and identifies
2,4-pyridine-dicarboxylic acid as an in vitro and in cell inhibitor. Febs j, 2012. 279(11): p. 1905-14.
57. Zheng, H., et al., Jumonji domain-containing 6 (JMJD6) identified as a potential therapeutic target in ovarian cancer. Signal Transduct Target Ther, 2019. 4: p. 24.
58. Berman, H. M., et al., The Protein Data Bank. Nucleic Acids Res, 2000. 28(1): p. 235-

[symbol]

1. A JMJD6 targeting agent for use in the treatment or prevention of prostate cancer.

2. The JMJD6 targeting agent for use according to claim 1, wherein the JMJD6 targeting agent is a modulator of the catalytic activity and/or biological function of JMJD6 protein.

3. The JMJD6 targeting agent for use according to claim 1 or 2, wherein the JMJD6 targeting agent is a small molecule inhibitor, or an antibody or fragment thereof.

4. The JMJD6 targeting agent for use according to any of claims 1 to 3, wherein the JMJD6 targeting agent is a substrate or 2OG (2-oxoglutarate) or dioxygen co-substrate competitive inhibitor, a non-competitive inhibitor or an un-competitive inhibitor.

5. The JMJD6 targeting agent for use according to any of claims 1 to 4, wherein the JMJD6 targeting agent comprises a compound which targets the active site of JMJD6.

6. The JMJD6 targeting agent for use according to any of claims 1 to 5, wherein the JMJD6 targeting agent comprises a compound which is a mimic, variant or competitor of the 2OG (2-oxoglutarate) JMJD6 co-substrate.

7. The JMJD6 targeting agent for use according to claim 6, wherein the JMJD6 targeting agent comprises a pyridine-carboxylate derivative, or N-oxalyl amino acid derivative, or succinate derivative, or 2OG or 2-oxo acid derivative,

8. The JMJD6 targeting agent for use according to claim 6 or 7, wherein the JMJD6 targeting agent comprises pyridine-2,4-dicarboxylic acid.

9. The JMJD6 targeting agent for use according to claim 1, wherein the JMJD6 targeting agent is capable of inhibiting expression of the JMJD6 gene, optionally wherein the JMJD6 targeting agent is selected from an antisense oligonucleotide or a mediator of RNAi such as an siRNA, shRNA, or other nucleotide molecule.

10. The JMJD6 targeting agent for use according to any of claims 1 to 9, wherein the JMJD6 targeting agent reduces the production of splice variants of the androgen receptor, preferably wherein the targeting agent reduces production of the AR-V7 splice variant.

11. The JMJD6 targeting agent for use according to any of claims 1 to 10, wherein the prostate cancer is selected from acinar adenocarcinoma, ductal adenocarcinoma, transitional cell carcinoma (urothelial carcinoma), squamous cell prostate cancer, small cell prostate cancer, large cell prostate cancer, mucinous adenocarcinoma, signet cell prostate cancer, basal cell prostate cancer, leiomyosarcoma, rhabdomyosarcoma, endocrine-resistant prostate cancer, or castration-resistant prostate cancer.

12. The JMJD6 targeting agent for use according to any preceding claims wherein the JMJD6 targeting agent is used in combination with a further anti-cancer therapy.

13. The JMJD6 targeting agent for use according to claim 12, wherein the further anti-cancer therapy is selected from radiotherapy, chemotherapy, surgery, immunotherapy, checkpoint inhibitors, hormone therapy or gene therapy.

14. The JMJD6 targeting agent for use according to claim 12 or 13, wherein the further anti-cancer therapy is selected from radium-223, docetaxel, sipuleucel-T, cabazitaxel, mitoxantrone, bicalutamide, ketoconazole, and/or corticosteroids or an anti-androgen therapy, such as abiraterone/ abiraterone acetate, enzalutamide or apalutamide.

15. The JMJD6 targeting agent for use according to any of claims 12 to 14 wherein the further anti-cancer therapy is administered simultaneously, sequentially or separately, with the JMJD6 targeting compound.

16. A pharmaceutical composition comprising a JMJD6 targeting agent according to any preceding claim, optionally further comprising one or more additional active agents, a pharmaceutically acceptable carrier, diluent, excipient or adjuvant.

17. The JMJD6 targeting agent for use according to claims 1 to 15, or pharmaceutical composition according to claim 16 wherein the JMJD6 targeting compound or the pharmaceutical composition is administered intravenously, subcutaneously, intramuscularly, or intradermally.

18. A kit comprising a JMJD6 targeting agent according to any of claims 1 to 15 or a pharmaceutical composition according to claim 16 and instructions for use.

19. A method of diagnosing or prognosing prostate cancer, comprising
a. obtaining a biological sample,
b. determining the level of JMJD6 in the sample; e.g. gene expression; wherein an increased level of JMJD6 compared to a reference sample indicates a poor prognosis.

20. The method according to claim 19, wherein expression level of JMJD6 is detected using a technique selected from reverse transcriptase-polymerase chain reaction (RT-PCR) methods, quantitative real-time PCR (qPCR), microarray, RNA sequencing (RNA-Seq), next generation RNA sequencing (deep sequencing), gene expression analysis by massively parallel signature sequencing (MPSS), or transcriptomics.

21. The method according to claims 19 to 20 wherein the method is for the diagnosis or prognosis of acinar adenocarcinoma, ductal adenocarcinoma, transitional cell carcinoma (urothelial carcinoma), squamous cell prostate cancer, small cell prostate cancer, large cell prostate cancer, mucinous adenocarcinoma, signet cell prostate cancer, basal cell prostate cancer, leiomyosarcoma, rhabdomyosarcoma, endocrine-resistant prostate cancer, or castration-resistant prostate cancer.

22. A method of inhibiting androgen receptor splicing comprising contacting a cell with a JMJD6 targeting agent.

23. A method of monitoring the therapeutic efficacy of a prostate cancer treatment, comprising; determining the level of JMJD6 prior to administration of the therapy and determining the level of JMJD6 after administration of the therapy.

24. A pharmaceutical composition comprising an androgen therapy and a JMJD6 targeting agent.

25. A method of identifying a JMJD6 targeting agent, comprising contacting a cell with a compound and determining the level of androgen receptor splicing.

26. A compound obtained or obtainable by the method according to claim 25.


SEQUENCE LISTING (4 sequences)

SEQ ID NO: 1 - DNA, 1245 bp, Homo sapiens
atgaaccacaagagcaagaagcgcatccgcgaggccaagcggagtgcgcggccggagctcaaggactcgctggattggacccggcacaactactacgagagcttctcgctgagcccggcggccgtggcggataacgtggaaagggcagatgctttacagctgtctgtggaagaatttgtggagcggtatgaaagaccttacaagcccgtggttttgttgaatgcgcaagagggctggtctgcgcaggagaaatggactctggagcgcctaaaaaggaaatatcggaaccagaagttcaagtgtggtgaggataacgatggctactcagtgaagatgaagatgaaatactacatcgagtacatggagagcactcgagatgatagtcccctttacatctttgacagcagctatggtgaacaccctaaaagaaggaaacttttggaagactacaaggtgccaaagtttttcactgatgaccttttccagtatgctggggagaagcgcaggcccccttacaggtggtttgtgatggggccaccacgctccggaactgggattcacatcgaccctctgggaaccagtgcctggaatgccttagttcagggccacaagcgctggtgcctgtttcctaccagcactcccagggaactcatcaaagtgacccgagacgaaggagggaaccagcaagacgaagctattacctggtttaatgttatttatccccggacacagcttccaacctggccacctgaattcaaacccctggaaatcttacaaaaaccaggagagactgtctttgtaccaggaggctggtggcatgttgtcctcaatctcgacactactatcgccatcacccaaaattttgccagcagcaccaacttccctgtggtatggcacaagacggtaagagggagaccaaagttatcaaggaaatggtataggattttgaagcaagagcaccccgagttggcagtcctcgcagactcggttgaccttcaggagtccacagggatagcttccgacagctccagcgactcttccagctcctccagctccagttcgtcagactccgactcagagtgcgagtctggatccgagggcgatgggacagtgcaccgcaggaagaagaggaggacgtgcagcatggtgggaaacggggacaccacctcccaggacgactgtgtcagcaaagagcgcagctcctccaggattagggacacttgtggaggccgggctcacccctga

SEQ ID NO: 2 - protein, 414 aa, Homo sapiens
MNHKSKKRIREAKRSARPELKDSLDWTRHNYYESFSLSPAAVADNVERADALQLSVEEFVERYERPYKPVVLLNAQEGWSAQEKWTLERLKRKYRNQKFKCGEDNDGYSVKMKMKYYIEYMESTRDDSPLYIFDSSYGEHPKRRKLLEDYKVPKFFTDDLFQYAGEKRRPPYRWFVMGPPRSGTGIHIDPLGTSAWNALVQGHKRWCLFPTSTPRELIKVTRDEGGNQQDEAITWFNVIYPRTQLPTWPPEFKPLEILQKPGETVFVPGGWWHVVLNLDTTIAITQNFASSTNFPVVWHKTVRGRPKLSRKWYRILKQEHPELAVLADSVDLQESTGIASDSSSDSSSSSSSSSSDSDSECESGSEGDGTVHRRKKRRTCSMVGNGDTTSQDDCVSKERSSSRIRDTCGGRAHP

SEQ ID NO: 3 - DNA, 1212 bp, Homo sapiens
atgaaccacaagagcaagaagcgcatccgcgaggccaagcggagtgcgcggccggagctcaaggactcgctggattggacccggcacaactactacgagagcttctcgctgagcccggcggccgtggcggataacgtggaaagggcagatgctttacagctgtctgtggaagaatttgtggagcggtatgaaagaccttacaagcccgtggttttgttgaatgcgcaagagggctggtctgcgcaggagaaatggactctggagcgcctaaaaaggaaatatcggaaccagaagttcaagtgtggtgaggataacgatggctactcagtgaagatgaagatgaaatactacatcgagtacatggagagcactcgagatgatagtcccctttacatctttgacagcagctatggtgaacaccctaaaagaaggaaacttttggaagactacaaggtgccaaagtttttcactgatgaccttttccagtatgctggggagaagcgcaggcccccttacaggtggtttgtgatggggccaccacgctccggaactgggattcacatcgaccctctgggaaccagtgcctggaatgccttagttcagggccacaagcgctggtgcctgtttcctaccagcactcccagggaactcatcaaagtgacccgagacgaaggagggaaccagcaagacgaagctattacctggtttaatgttatttatccccggacacagcttccaacctggccacctgaattcaaacccctggaaatcttacaaaaaccaggagagactgtctttgtaccaggaggctggtggcatgttgtcctcaatctcgacactactatcgccatcacccaaaattttgccagcagcaccaacttccctgtggtatggcacaagacggtaagagggagaccaaagttatcaaggaaatggtataggattttgaagcaagagcaccccgagttggcagtcctcgcagactcggttgaccttcaggagtccacagggatagcttccgacagctccagcgactcttccagctcctccagctccagttcgtcagactccgactcagagtgcgagtctggatccgagggcgatgggacagtgcaccgcaggaagaagaggaggacgtgcagcatggtgggaaacggggacaccacctcccaggacgactgtgtcagcaaagagcgcagctcctccaggtga

SEQ ID NO: 4 - protein, 403 aa, Homo sapiens
MNHKSKKRIREAKRSARPELKDSLDWTRHNYYESFSLSPAAVADNVERADALQLSVEEFVERYERPYKPVVLLNAQEGWSAQEKWTLERLKRKYRNQKFKCGEDNDGYSVKMKMKYYIEYMESTRDDSPLYIFDSSYGEHPKRRKLLEDYKVPKFFTDDLFQYAGEKRRPPYRWFVMGPPRSGTGIHIDPLGTSAWNALVQGHKRWCLFPTSTPRELIKVTRDEGGNQQDEAITWFNVIYPRTQLPTWPPEFKPLEILQKPGETVFVPGGWWHVVLNLDTTIAITQNFASSTNFPVVWHKTVRGRPKLSRKWYRILKQEHPELAVLADSVDLQESTGIASDSSSDSSSSSSSSSSDSDSECESGSEGDGTVHRRKKRRTCSMVGNGDTTSQDDCVSKERSSSR